(19) United States
(12) Patent Application Publication
(10) Pub. No.: US 2019/0298202 A1
(43) Pub. Date: Oct. 3, 2019

(54) MAGNETOCARDIOGRAPHIC MEASUREMENT APPARATUS, CALIBRATION METHOD, AND RECORDING MEDIUM HAVING RECORDED THEREON CALIBRATION PROGRAM

(71) Applicant: Asahi Kasei Microdevices Corporation, Tokyo (JP)

(72) Inventors: Takenobu NAKAMURA, Tokyo (JP); Shigeki OKATAKE, Tokyo (JP); Yoshitaka MORIYASU, Tokyo (JP)

(21) Appl. No.: 16/365,689

(22) Filed: Mar. 27, 2019

(30) Foreign Application Priority Data
Mar. 28, 2018 (JP) 2018-063190
Jan. 22, 2019 (JP) 2019-008593

(51) Int. Cl. A61B 5/04; G01R 33/09; A61B 5/00

(52) U.S. Cl. CPC A61B 5/04007; A61B 5/7225; G01R 33/09

(57) ABSTRACT

A highly precise, and simple and easy calibration function of a magnetocardiographic measurement apparatus is provided. A magnetocardiographic measurement apparatus includes: a magnetic sensor array; and a magnetic field acquiring unit acquiring environmental magnetic field measurement data measured by the array in response to the array being turned such that the array faces a plurality of directions in an environmental magnetic field; a calibration parameter calculating unit using the environmental magnetic field measurement data to calculate a calibration parameter for calibrating measurement data measured by the array in magnetocardiographic measurement of a subject; a calibration parameter storage unit storing the calibration parameter; a calibration calculating unit using the calibration parameter to calibrate the measurement data; and a data output unit outputting the measurement data. The array has a plurality of magnetic sensor cells arrayed three-dimensionally, and each capable of sensing a magnetic field in three axial directions.

The contents of the following Japanese patent application(s) are incorporated herein by reference:
2018-063190 filed in JP on Mar. 28, 2018; and
2019-008593 filed in JP on Jan. 22, 2019

### BACKGROUND

### 1. Technical Field

The present invention relates to a magnetocardiographic measurement apparatus, a calibration method, and a calibration program.

### 2. Related Art

Magnetocardiographs using superconducting quantum interference devices (SQUIDs) have conventionally been put into practical use (Patent Literature 1). The SQUID magnetocardiographs are expensive, and require cooling using liquid helium. Accordingly, magnetocardiographs using magneto-resistance effect (“MR” or “xMR”) devices such as tunnel magneto-resistance effect (TMR) devices or giant magneto-resistance effect (GMR) devices are under development (Patent Literatures 2, 3).
Patent Literature 1: Japanese Patent Application Publication No. 2001-87237
Patent Literature 2: Japanese Patent Application Publication No. 2012-152514
Patent Literature 3: WO 2017/209273

### SUMMARY

Since MR sensors use ferromagnetic materials, their magnetic characteristics vary significantly if they are exposed to magnetic fields when magnets are brought close to them after product shipment or for other reasons.

In order to solve the above-mentioned drawbacks, a first aspect of the present invention provides a magnetocardiographic measurement apparatus. The magnetocardiographic measurement apparatus may include a magnetic sensor array. The magnetocardiographic measurement apparatus may include a magnetic field acquiring unit that acquires environmental magnetic field measurement data measured by the magnetic sensor array in response to the magnetic sensor array being turned such that the magnetic sensor array faces a plurality of directions in an environmental magnetic field. The magnetocardiographic measurement apparatus may include a calibration parameter calculating unit that uses the environmental magnetic field measurement data to calculate a calibration parameter for calibrating measurement data measured by the magnetic sensor array in magnetocardiographic measurement of a subject. The magnetocardiographic measurement apparatus may include a calibration parameter storage unit that stores the calculated calibration parameter. The magnetocardiographic measurement apparatus may include a calibration calculating unit that uses the stored calibration parameter to calibrate the measurement data. The magnetocardiographic measurement apparatus may include a data output unit that outputs the calibrated measurement data. The magnetic sensor array may have a plurality of magnetic sensor cells that are arrayed three-dimensionally, and are each capable of sensing a magnetic field in three axial directions.

The magnetic field acquiring unit may acquire the environmental magnetic field measurement data on-site.

If first magnetocardiographic measurement is performed on a subject, the magnetic field acquiring unit may acquire the environmental magnetic field measurement data to be used in calibration of measurement data measured in the first magnetocardiographic measurement by the magnetic sensor array.

The magnetic field acquiring unit may acquire the environmental magnetic field measurement data measured before and/or after the first magnetocardiographic measurement.

The magnetic field acquiring unit may acquire the environmental magnetic field measurement data measured: after magnetocardiographic measurement of a previous subject and before the first magnetocardiographic measurement; and/or after the first magnetocardiographic measurement, and before magnetocardiographic measurement of a succeeding subject.

The magnetic field acquiring unit may acquire first environmental magnetic field measurement data measured before the first magnetocardiographic measurement, and second environmental magnetic field measurement data measured after the first magnetocardiographic measurement. The calibration parameter calculating unit may use the first environmental magnetic field measurement data to calculate the calibration parameter. The magnetocardiographic measurement apparatus may further include a judging unit that uses the second environmental magnetic field measurement data to judge validity of measurement data measured in the first magnetocardiographic measurement by the magnetic sensor array.

The second environmental magnetic field measurement data may have a smaller number of data elements than the first environmental magnetic field measurement data does.

The magnetocardiographic measurement apparatus may further include a drive unit that alters an orientation of the magnetic sensor array. The magnetic field acquiring unit may acquire the environmental magnetic field measurement data in response to the magnetic sensor array being turned by the drive unit such that the magnetic sensor array faces the plurality of directions.

The drive unit may alter the orientation of the magnetic sensor array continuously. The magnetic field acquiring unit may sample a data element in the environmental magnetic field measurement data at predetermined timing while the orientation of the magnetic sensor array is being altered.

The drive unit may alter a zenith angle and an azimuth angle of the magnetic sensor array.

The drive unit may alter the orientation of the magnetic sensor array relative to a head of the magnetocardiographic measurement apparatus, the head being configured to make the magnetic sensor array face a subject.

The drive unit may alter the orientation of the magnetic sensor array after the magnetic sensor array is detached from a head of the magnetocardiographic measurement apparatus, the head being configured to make the magnetic sensor array face a subject.

A second aspect of the present invention provides a calibration method by which a magnetocardiographic measurement apparatus calibrates measurement data in magnetocardiographic measurement. The calibration method may include acquiring, by the magnetocardiographic measurement apparatus, environmental magnetic field measurement data measured by a magnetic sensor array in response to the magnetic sensor array being turned such that the magnetic sensor array faces a plurality of directions in an environmental magnetic field, the magnetic sensor array having a plurality of magnetic sensor cells that are arrayed three-dimensionally, and are each capable of sensing a magnetic field in three axial directions. The calibration method may include calculating, by the magnetocardiographic measurement apparatus using the environmental magnetic field measurement data, a calibration parameter for calibrating measurement data measured by the magnetic sensor array in magnetocardiographic measurement of a subject. The calibration method may include storing, in the magnetocardiographic measurement apparatus, the calculated calibration parameter. The calibration method may include calibrating, by the magnetocardiographic measurement apparatus, the measurement data using the stored calibration parameter.

In the acquisition of the environmental magnetic field measurement data, if first magnetocardiographic measurement is performed on a subject, the magnetocardiographic measurement apparatus may acquire the environmental magnetic field measurement data to be used in calibration of measurement data measured in the first magnetocardiographic measurement by the magnetic sensor array.

In the acquisition of the environmental magnetic field measurement data, the magnetocardiographic measurement apparatus may acquire the environmental magnetic field measurement data measured before and/or after the first magnetocardiographic measurement.

The calibration method may further include altering an orientation of the magnetic sensor array by the magnetocardiographic measurement apparatus. In the acquisition of the environmental magnetic field measurement data, the magnetocardiographic measurement apparatus may acquire the environmental magnetic field measurement data in response to the magnetic sensor array being turned such that the magnetic sensor array faces the plurality of directions.

In the alteration of the orientation of the magnetic sensor array, the magnetocardiographic measurement apparatus may alter the orientation of the magnetic sensor array continuously. In the acquisition of the environmental magnetic field measurement data, the magnetocardiographic measurement apparatus may sample a data element in the environmental magnetic field measurement data at predetermined timing while the orientation of the magnetic sensor array is being altered.

In the alteration of the orientation of the magnetic sensor array, the magnetocardiographic measurement apparatus may alter a zenith angle and an azimuth angle of the magnetic sensor array.

A third aspect of the present invention provides a calibration program. The calibration program may be executed by a computer. The calibration program may cause the computer to function as a recording medium having recorded thereon a calibration program that, upon being executed by a computer, causes the computer to function as a magnetic field acquiring unit that acquires environmental magnetic field measurement data measured by a magnetic sensor array in response to the magnetic sensor array being turned such that the magnetic sensor array faces a plurality of directions in an environmental magnetic field, the magnetic sensor array having a plurality of magnetic sensor cells that are arrayed three-dimensionally, and are each capable of sensing a magnetic field in three axial directions. The calibration program may cause the computer to function as a calibration parameter calculating unit that uses the environmental magnetic field measurement data to calculate a calibration parameter for calibrating measurement data measured by the magnetic sensor array in magnetocardiographic measurement of a subject. The calibration program may cause the computer to function as a calibration parameter storage unit that stores the calculated calibration parameter. The calibration program may cause the computer to function as a calibration calculating unit that uses the stored calibration parameter to calibrate the measurement data.

The summary clause does not necessarily describe all necessary features of the embodiments of the present invention. The present invention may also be a sub-combination of the features described above.

### BRIEF DESCRIPTION OF THE DRAWINGS

FIG. 1 illustrates the configuration of a magnetic field measuring apparatus 10 according to the present embodiment.

FIG. 2 illustrates the configuration of a magnetic sensor unit 110 according to the present embodiment.

FIG. 3 illustrates exemplary input/output characteristics of a magnetic sensor having a magneto-resistance device according to the present embodiment.

FIG. 4 illustrates an exemplary configuration of a sensor unit 400 according to the present embodiment.

FIG. 5 illustrates exemplary input/output characteristics of the sensor unit 400 according to the present embodiment.

FIG. 6 illustrates the configurations of a magnetic sensor array 210, a sensor data collecting unit 230, and a sensor data processing unit 600 according to the present embodiment.

FIG. 7 illustrates an exemplary magnetocardiographic measurement flow for the magnetic field measuring apparatus 10 according to the present embodiment.

FIG. 8 illustrates an exemplary calibration flow for the magnetic field measuring apparatus 10 according to the present embodiment.

FIG. 9 illustrates exemplary ellipsoid fitting and conversion into a sphere in calibration by the magnetic field measuring apparatus 10 according to the present embodiment.

FIG. 10 illustrates exemplary measurement data before conversion into a sphere by the magnetic field measuring apparatus 10 according to the present embodiment.

FIG. 11 illustrates a projection, on the YZ plane, of the measurement data before conversion into a sphere illustrated in FIG. 10.

FIG. 12 illustrates exemplary measurement data after conversion into a sphere by the magnetic field measuring apparatus 10 according to the present embodiment.

FIG. 13 illustrates a projection, on the YZ plane, of the measurement data after conversion into a sphere illustrated in FIG. 12.

FIG. 14 shows an example of a computer 2200 in which aspects of the present invention may be wholly or partly embodied.

### DESCRIPTION OF EXEMPLARY EMBODIMENTS

Hereinafter, (some) embodiment(s) of the present invention will be described. The embodiment(s) do(es) not limit the invention according to the claims, and all the combinations of the features described in the embodiment(s) are not necessarily essential to means provided by aspects of the invention.

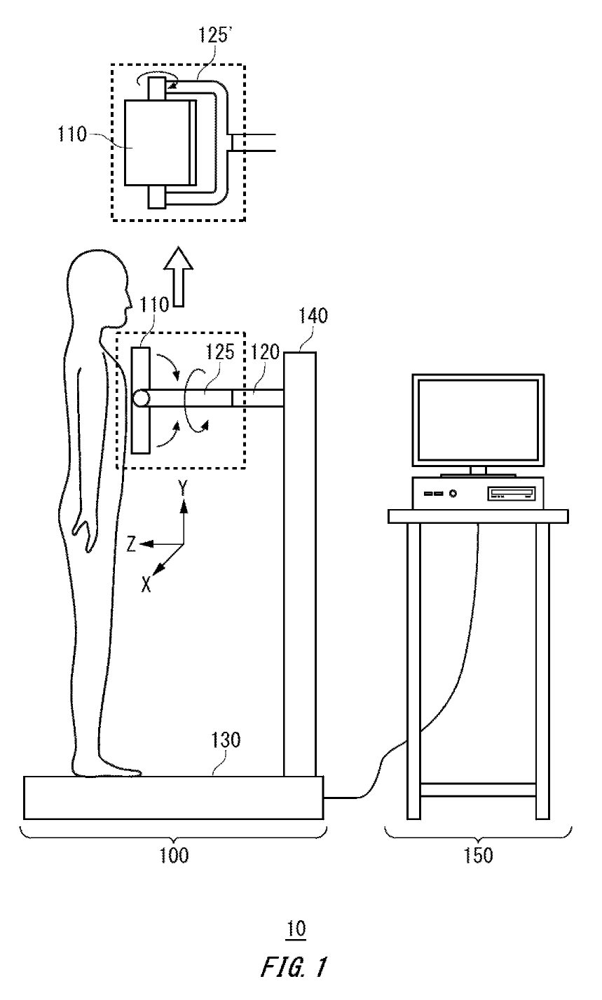

FIG. 1 illustrates the configuration of a magnetic field measuring apparatus 10 according to the present embodiment. The magnetic field measuring apparatus 10 uses an MR device to measure a magnetic field (referred to as a biomagnetic field) generated by an electrical activity of a living body (a human body, etc.). The magnetic field measuring apparatus 10 has a highly precise, and simple and easy calibration function, and thereby can measure a biomagnetic field highly precisely without being installed in a shield room.

The magnetic field measuring apparatus 10 is an exemplary magnetocardiographic measurement apparatus, and measures a magnetic field (referred to as a cardiac magnetic field) generated by an electrical activity of the heart of a living body. In the present embodiment, the magnetic field measuring apparatus 10 is used for measuring a cardiac magnetic field of a subject. Instead, the magnetic field measuring apparatus 10 may be used for measuring a cardiac magnetic field of a non-human living body. The magnetic field measuring apparatus 10 includes a main-body portion 100, and an information processing unit 150. The main-body portion 100 is a component for sensing a cardiac magnetic field of a subject, and has a magnetic sensor unit 110, a head 120, a drive unit 125, a base portion 130, and a pole portion 140.

The magnetic sensor unit 110 is arranged at such a position that the magnetic sensor unit 110 faces the heart of a subject in his/her chest portion at the time of magnetocardiographic measurement, and senses a cardiac magnetic field of the subject. The head 120 supports the magnetic sensor unit 110, and makes the magnetic sensor unit 110 face a subject. The drive unit 125 is provided between the magnetic sensor unit 110 and the head 120, and alters the orientation of the magnetic sensor unit 110 relative to the head 120 when calibration is performed. The drive unit 125 according to the present embodiment includes: a first actuator capable of rotating the magnetic sensor unit 110 by 360 degrees about the Z axis in the figure; and a second actuator that rotates the magnetic sensor unit 110 about an axis orthogonal to the Z axis (the X axis in the state illustrated in the figure). The drive unit 125 uses the first actuator and second actuator to alter the azimuth angle and zenith angle of the magnetic sensor unit 110. Here, for example, the azimuth angle of the magnetic sensor unit 110 may be an angle by which a plane of the magnetic sensor unit 110 rotates about an axis (the Z axis in the figure) coinciding with the direction of zenith which is defined as the direction pointing to the chest of a subject, and the zenith angle of the magnetic sensor unit 110 may be an angle that the plane of the magnetic sensor unit 110 forms with the axis coinciding with the direction of zenith. As illustrated as a drive unit 125′ in the figure, the drive unit 125 has a Y-shape when seen in the Y-axis direction in the figure, and the second actuator can rotate the magnetic sensor unit 110 by 360 degrees about the X axis in the figure.

The base portion 130 is a base supporting other components of the main-body portion 100, and is the platform which the subject gets on at the time of magnetocardiographic measurement in the present embodiment. The pole portion 140 supports the head 120 at the height of the chest portion of the subject. The pole portion 140 may be expandable and contractable in the vertical direction such that the height of the magnetic sensor unit 110 is adjusted to match the height of the chest portion of the subject.

The information processing unit 150 is a component for processing data acquired through measurement by the main-body portion 100, and outputting the data on a display, by printing, or in another manner. The information processing unit 150 may be a computer such as a PC (personal computer), a tablet computer, a smartphone, a work station, a server computer, or a general purpose computer, or may be a computer system constituted by a plurality of interconnected computers. Instead, the information processing unit 150 may be a specialized computer designed for information processing in magnetocardiographic measurement, and may be specialized hardware realized by a specialized circuit.

The magnetic field measuring apparatus 10 according to the present embodiment has a configuration that enables magnetocardiographic measurement while the subject is in an upright position so as to provide simple and easy magnetocardiographic measurement with reduced mental burden on the subject. Instead, the magnetic field measuring apparatus 10 may have a configuration that enables magnetocardiographic measurement while the subject is in a recumbent position so as to enhance the precision of magnetocardiographic measurement by more stably fixing the position of the subject.

In addition, the drive unit 125 according to the present embodiment alters the orientation of the magnetic sensor unit 110 while the magnetic sensor unit 110 is supported by the head 120. Instead, the drive unit 125 may include a robot arm or the like that is left unused and does not contact the magnetic sensor unit 110 and the head 120 at the time of magnetocardiographic measurement. In such a case, the drive unit 125 may alter the orientation of the magnetic sensor unit 110 after the magnetic sensor unit 110 is detached from the head 120 at the time of calibration, or may alter the orientation of a magnetic sensor array 210 described below which constitutes the magnetic sensor unit 110 after the magnetic sensor array 210 is detached.

Note that the magnetic field measuring apparatus 10 according to the present embodiment is a magnetocardiographic measurement apparatus, for example, but instead the magnetic field measuring apparatus 10 may be configured to measure a biomagnetic field other than a cardiac magnetic field produced in a living body, such as a cerebral magnetic field. The structures of the base portion 130 and pole portion 140, the orientation of the head 120, and the like may be altered such that they have suitable structures/arrangements depending on a to-be-measured portion of a living body.

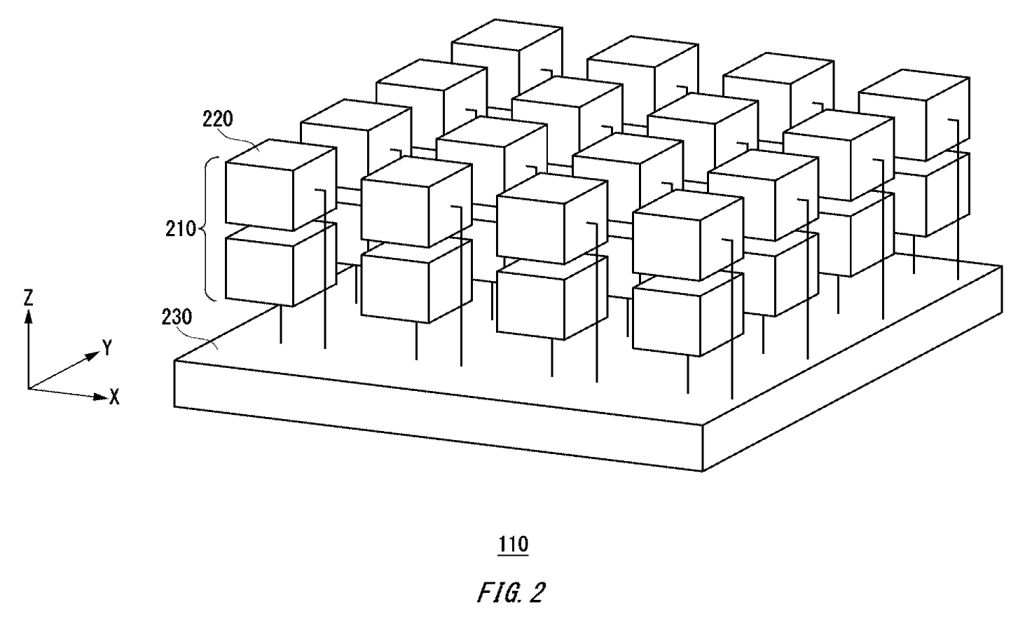

FIG. 2 illustrates the configuration of the magnetic sensor unit 110 according to the present embodiment. The magnetic sensor unit 110 has the magnetic sensor array 210, and a sensor data collecting unit 230. The magnetic sensor array 210 has a configuration in which a plurality of magnetic sensor cells 220 are arrayed one-dimensionally, two-dimensionally or three-dimensionally. In this figure, the magnetic sensor array 210 includes magnetic sensor cells 220 that are arrayed in four rows in the X direction, four rows in the Y direction, and two rows in the Z direction. Here, the position of each magnetic sensor cell 220 in the magnetic sensor array 210 is represented by a set [i, j, k] of the position i in the X direction, the position j in the Y direction, and the position k in the Z direction. Here, i is an integer satisfying the relationship 0≤i≤Nx−1 (Nx is the number of rows of magnetic sensor cells 220 in the X direction), j is an integer satisfying the relationship 0≤j≤Ny−1 (Ny is the number of rows of magnetic sensor cells 220 in the Y direction), and k is an integer satisfying the relationship 0≤k≤Nz−1 (Nz is the number of rows of magnetic sensor cells 220 in the Z direction).

The sensor data collecting unit 230 is electrically connected to the plurality of magnetic sensor cells 220 included in the magnetic sensor array 210, collects sensor data (sensing signals) from the plurality of magnetic sensor cells 220, and supplies the sensor data to the information processing unit 150.

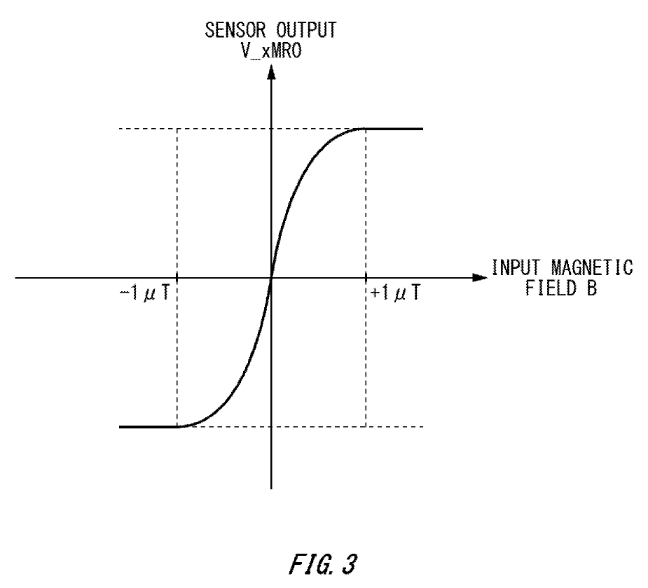

FIG. 3 illustrates exemplary input/output characteristics of a magnetic sensor having a magneto-resistance device according to the present embodiment. In this figure, the horizontal axis corresponds to the magnitude B of an input magnetic field input to the magnetic sensor, and the vertical axis corresponds to the magnitude V_xMR0 of a signal acquired through sensing by the magnetic sensor. The magnetic sensor has, for example, a giant magneto-resistance (GMR: Giant Magneto-Resistance) device, a tunnel magneto-resistance (TMR: Tunnel Magneto-Resistance) device, or the like, and senses the magnitude of a magnetic field in a predetermined one axial direction.

The magnetosensitivity of such a magnetic sensor, which is indicated by the slope of the magnitude V_xMR0 of the sensing signal relative to the magnitude B of the input magnetic field, is high, and the magnetic sensor can sense a magnetic field as minute as about 10 pT. On the other hand, with the magnetic sensor, the magnitude V_xMR0 of the sensing signal reaches the saturation level when the absolute value of the magnitude B of the input magnetic field is about 1 μT, for example, and so the magnetic sensor has favorable linear input/output characteristics only in a small range. In view of this, the linear characteristics of such a magnetic sensor can be improved by adding a closed loop that produces a feedback magnetic field in the magnetic sensor. Such a magnetic sensor is explained next.

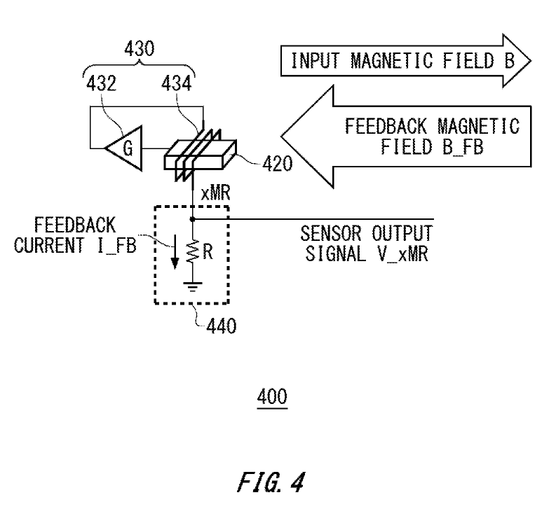

FIG. 4 illustrates an exemplary configuration of a sensor unit 400 according to the present embodiment. The sensor unit 400 is provided inside each of the plurality of magnetic sensor cells 220, and has a magnetic sensor 420, a magnetic field generating unit 430, and an output unit 440. Note that part of the sensor unit 400 (e.g., an amplifier circuit 432, and the output unit 440) may be provided not in the magnetic sensor cell 220, but in the sensor data collecting unit 230.

Similar to the magnetic sensor explained with reference to FIG. 3, the magnetic sensor 420 has a magneto-resistance effect device such as a GMR device or a TMR device. For example, the positive direction of a magnetosensitive axis is defined as the +X direction. The magnetic sensor 420 may be formed to have an increased resistance if a magnetic field in the +X direction is input, and have a decreased resistance if a magnetic field in the −X direction is input. That is, the magnitude B of the magnetic field input to the magnetic sensor 420 can be sensed by observing changes of the resistance of the magnetic sensor 420. For example, the magnetosensitivity of the magnetic sensor 420 is defined as S. A result of sensing of the magnitude B of the input magnetic field by the magnetic sensor 420 can be calculated by S×B. Note that, for example, a power supply or the like is connected to the magnetic sensor 420, and the magnetic sensor 420 outputs a voltage drop corresponding to a change of the resistance as a result of sensing of an input magnetic field.

The magnetic field generating unit 430 applies, to the magnetic sensor 420, a feedback magnetic field to reduce the input magnetic field sensed by the magnetic sensor 420. The magnetic field generating unit 430 operates, for example, to produce a feedback magnetic field B_FB which is in the opposite direction to the magnetic field with the magnitude B input to the magnetic sensor 420, and has an absolute value which is approximately the same as that of the input magnetic field to thereby cancel the input magnetic field. The magnetic field generating unit 430 includes the amplifier circuit 432, and a coil 434.

The amplifier circuit 432 outputs, as a feedback current I_FB, a current corresponding to a result of sensing of the input magnetic field by the magnetic sensor 420. The amplifier circuit 432 includes a transconductance amplifier, for example, and outputs the feedback current I_FB corresponding to an output voltage of the magnetic sensor 420. For example, the voltage-current conversion coefficient of the amplifier circuit 432 is defined as G The feedback current I_FB can be calculated by G×S×B. The coil 434 produces a feedback magnetic field B_FB corresponding to the feedback current I_FB. The coil 434 desirably produces a feedback magnetic field B_FB that is uniform over the entire magnetic sensor 420. For example, the coil coefficient of the coil 434 is defined as β. The feedback magnetic field B_FB can be calculated by β×I_FB. Here, since the feedback magnetic field B_FB is produced in such a direction that the input magnetic field with the magnitude B is cancelled, the magnetic field input to the magnetic sensor 420 is reduced to B-B_FB. Accordingly, the feedback current I_FB is represented by the following formula.

I_FB=G×S×(B−β×I_FB) [Formula 1]

By solving Formula 1 for the feedback current I_FB, the value of the feedback current I_FB in the steady state of the sensor unit 400 can be calculated. If the magnetosensitivity S of the magnetic sensor 420, and the voltage-current conversion coefficient G of the amplifier circuit 432 are sufficiently high and large, the following formula is derived from Formula 1.

$$I\_FB = \frac{G \times S \times B}{1 + G \times S \times β} ≅ \frac{B}{β} \qquad [ Formula 2 ]$$

The output unit 440 outputs an output signal with the magnitude V_xMR corresponding to the feedback current I_FB fed by the magnetic field generating unit 430 to produce the feedback magnetic field B_FB. The output unit 440 has a resistive device with a resistance R, for example, and outputs, as the output signal with the magnitude V_xMR, a voltage drop produced by the feedback current I_FB flowing through the resistive device. In this case, the magnitude V_xMR of the output signal is calculated according to Formula 2 as indicated by the following formula.

$$V\_xMR = R \times I\_FB = \frac{R \times B}{β} \qquad [ Formula 3 ]$$

In this manner, since the sensor unit 400 produces a feedback magnetic field to reduce a magnetic field input from the outside, the sensor unit 400 reduces a magnetic field substantially input to the magnetic sensor 420. Thereby, for example, even if a magneto-resistance device having the characteristics illustrated in FIG. 3 is used as the magnetic sensor 420, and the absolute value of the magnitude B of the input magnetic field exceeds 1 μT, the sensor unit 400 can prevent the magnitude V_xMR of the sensing signal from reaching the saturation level. The input/output characteristics of such a sensor unit 400 are explained next.

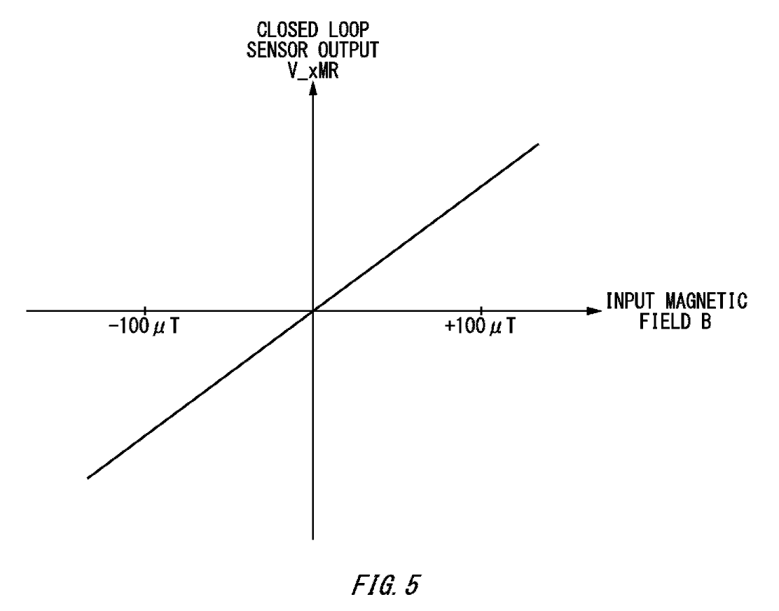

FIG. 5 illustrates exemplary input/output characteristics of the sensor unit 400 according to the present embodiment. In this figure, the horizontal axis corresponds to the magnitude B of an input magnetic field input to the sensor unit 400, and the vertical axis corresponds to the magnitude V_xMR of a signal acquired through sensing by the sensor unit 400. The sensor unit 400 has a high magnetosensitivity, and can sense a magnetic field as minute as about 10 pT. In addition, even if the absolute value of the magnitude B of the input magnetic field exceeds 100 μT, for example, the sensor unit 400 can maintain favorable linear characteristics of the magnitude V_xMR of the sensing signal.

That is, the sensor unit 400 according to the present embodiment is configured such that a result of sensing of the magnitude B of the input magnetic field by the sensor unit 400 has linear characteristics over a predetermined range of the magnitude B of the input magnetic field which corresponds to, for example, the absolute value of the magnitude B of the input magnetic field no greater than several hundreds μT. Using such a sensor unit 400, faint magnetic signals like cardiac magnetic field signals, for example, can be sensed simply and easily.

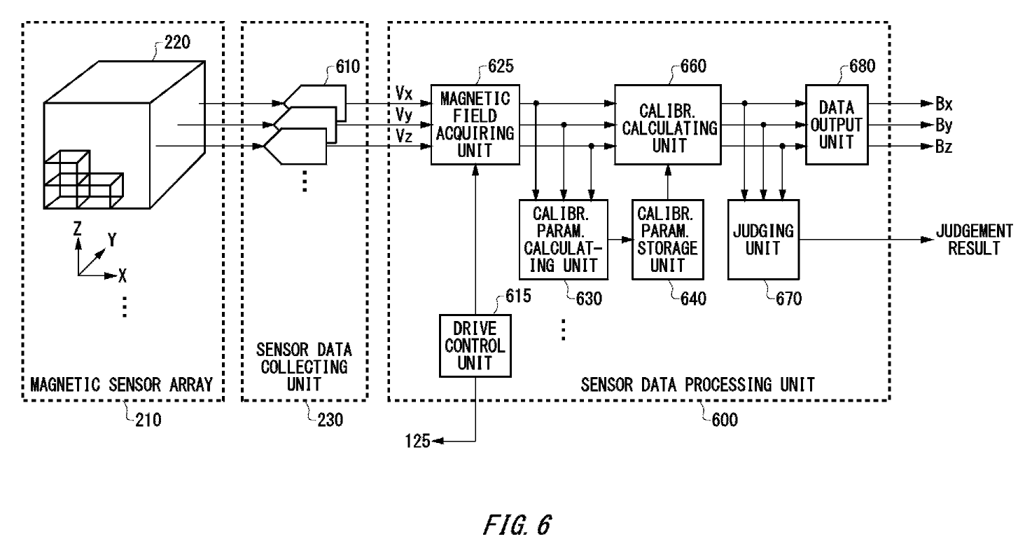

FIG. 6 illustrates the configurations of the magnetic sensor array 210, the sensor data collecting unit 230, and a sensor data processing unit 600 according to the present embodiment. In the figure, a drive control unit 615 is a configuration shared by the plurality of magnetic sensor cells 220. Other configurations that are provided in each magnetic sensor cell 220 are illustrated not entirely, but only those corresponding to one magnetic sensor cell 220 are illustrated.

The magnetic sensor array 210 has the plurality of magnetic sensor cells 220. In the present embodiment, each of the plurality of magnetic sensor cells 220 can sense a magnetic field in three axial directions. Each magnetic sensor cell 220 includes three sensor units 400 that measure magnetic fields in three axial directions which are the X direction, Y direction, and Z direction. Each sensor data collecting unit 230 is provided corresponding to one of the plurality of magnetic sensor cells 220, and converts an analog sensing signal (the magnitude V_xMR of the sensor output signal in FIG. 4) output by each sensor unit 400 of a corresponding magnetic sensor cell 220 into digital measurement data (Vx, Vy, Vz). Here, Vx, Vy, and Vz are measurements obtained by converting sensing signals from X direction, Y direction, and Z direction sensor units 400 into digital values (e.g., digital voltage values).

The sensor data processing unit 600 is provided in the information processing unit 150, processes measurement data from sensor data collecting units 230, and outputs the processed measurement data. The sensor data processing unit 600 has the drive control unit 615, and a magnetic field acquiring unit 625, a calibration parameter calculating unit 630, a calibration parameter storage unit 640, a calibration calculating unit 660, a judging unit 670, and a data output unit 680 that are provided correspond to each of the plurality of magnetic sensor cells 220. The drive control unit 615, the plurality of magnetic field acquiring units 625, the plurality of calibration parameter calculating units 630, the plurality of calibration parameter storage units 640, and the plurality of judging units 670 function, along with the drive unit 125, also as a calibrating apparatus that calculates calibration parameters for calibrating measurement data measured by the magnetic sensor array 210.

The drive control unit 615 drives the drive unit 125, and causes the drive unit 125 to alter the orientation of the magnetic sensor array 210. A magnetic field acquiring unit 625 is connected to an AD converter 610 via a wire between the sensor data collecting unit 230 and the information processing unit 150, and in response to the magnetic sensor array 210 being turned such that the magnetic sensor array 210 faces a plurality of directions in an environmental magnetic field (e.g., terrestrial magnetism), acquires environmental magnetic field measurement data measured by the magnetic sensor array 210. The magnetic field acquiring unit 625 may be configured using a flip-flop or the like that latches data elements (Vx, Vy, Vz) of measurement data at timing when the magnetic sensor array 210 is turned such that the magnetic sensor array 210 faces each direction of a plurality of directions. For example, while receiving environmental magnetic field measurement mode signals that are output when the drive control unit 615 is performing control of turning the magnetic sensor array 210 such that the magnetic sensor array 210 faces a plurality of directions in an environmental magnetic field, the magnetic field acquiring unit 625 may acquire measurement data in a predetermined sampling cycle. The magnetic field acquiring unit 625 may store, temporarily in the information processing unit 150, the environmental magnetic field measurement data acquired for the plurality of directions as computer files or the like. Instead, the magnetic field acquiring unit 625 may have any configuration capable of accepting measurement data such as an input terminal, an input port, an input wire or the like through which the measurement data is input. In addition, the magnetic field acquiring unit 625 acquires measurement data (referred to also as “magnetocardiographic data”) measured during magnetocardiographic measurement of a subject.

A calibration parameter calculating unit 630 is connected to a magnetic field acquiring unit 625, and uses environmental magnetic field measurement data acquired by the magnetic field acquiring unit 625 to calculate calibration parameters for calibrating measurement data measured by the magnetic sensor array 210 in magnetocardiographic measurement of a subject. A calibration parameter storage unit 640 is connected to a calibration parameter calculating unit 630, and stores calibration parameters calculated by the calibration parameter calculating unit 630. A calibration calculating unit 660 is connected to a magnetic field acquiring unit 625, and uses calibration parameters calculated by a calibration parameter calculating unit 630 and stored on a calibration parameter storage unit 640 to calibrate measurement data measured by the magnetic sensor array 210.

A judging unit 670 is connected to a calibration calculating unit 660, and uses second environmental magnetic field measurement data measured after magnetocardiographic measurement of a subject to judge the validity of measurement data from the magnetic sensor array measured in magnetocardiographic measurement. For example, if a calibration parameter calculating unit 630 uses first environmental magnetic field measurement data measured before magnetocardiographic measurement of a subject to calculate calibration parameters, and a calibration calculating unit 660 uses the calibration parameters to calibrate magnetocardiographic data, the judging unit 670 judges the validity of the magnetocardiographic data based on the degree of difference, from a targeted distribution, of measurement data obtained by calibrating, by the calibration calculating unit 660, second environmental magnetic field measurement data measured after magnetocardiographic measurement. That is, if a difference is observed between distributions of environmental magnetic field measurement data before and after magnetocardiographic measurement, the judging unit 670 judges that the validity is low, determining that a change occurred in the sensitivity of the magnetic sensor array 210 or the like, and magnetocardiographic measurement is inaccurate. The judging unit 670 may output a result of judgement about validity by adding the result to magnetocardiographic data output by the data output unit 680.

A data output unit 680 outputs calibrated measurement data. For example, the data output unit 680 may store calibrated measurement data in the information processing unit 150, in an external computer, in an external storage, and/or the like as computer files or the like, and may use the measurement data to generate an image indicating a magnetocardiographic measurement result, and display the image on a display device provided to the information processing unit 150.

Main points of calibration of measurement data by the sensor data processing unit 600 illustrated above are as follows. An input magnetic field input to a magnetic sensor cell 220 at a position [i, j, k] is defined as B (Bx, By, Bz), and a sensing result of triaxial magnetic sensors, an X-sensor unit 400, a Y-sensor unit 400, and a Z-sensor unit 400, is defined as V (Vx, Vy, Vz). The magnetic sensor characteristics of the triaxial magnetic sensors are defined as a matrix S. In this case, the following formula holds true.

$$( \begin{matrix}Vx \\ Vy \\ Vz\end{matrix} ) = S ( \begin{matrix}Bx \\ By \\ Bz\end{matrix} ) + ( \begin{matrix}Vos , x \\ Vos , y \\ Vos , z\end{matrix} ) = ( \begin{matrix}Sxx & Sxy & Sxz \\ Syx & Syy & Syz \\ Szx & Szy & Szz\end{matrix} ) ( \begin{matrix}Bx \\ By \\ Bz\end{matrix} ) + ( \begin{matrix}Vos , x \\ Vos , y \\ Vos , z\end{matrix} ) \qquad [ Formula 4 ]$$

Here, Sxx, Syy, and Szz represent the sensitivities of the X-sensor unit 400, Y-sensor unit 400, and Z-sensor unit 400 in the main axis directions (also called sensitive directions or input directions), respectively. Sxy, Sxz, Syx, Syz, Szx, and Szy represent sensitivities in other axial directions, and these are called cross axis sensitivities, which may mean sensitivities along axis perpendicular to input axis, and these sensitivities cause non-orthogonal errors in measurements. In addition, “Vos, x”, “Vos, y”, and “Vos, z” represent offsets of the X-sensor unit 400, Y-sensor unit 400, and Z-sensor unit 400, respectively.

Since a result of sensing of an input magnetic field by each of the sensor units 400 has linear characteristics over a range of the input magnetic field which it is supposed to be sensitive to, each element of the matrix S becomes an approximately constant coefficient not related to the magnitude B of the input magnetic field. In addition, even if a sensor unit 400 has a cross-axis sensitivity, each element of the matrix S becomes an approximately constant coefficient not related to the magnitude B of the input magnetic field as long as a result of sensing by the sensor unit 400 has linear characteristics.

Accordingly, using the inverse matrix 5′ of the matrix S and the offsets (Vos, x, Vos, y, Vos, z), the calibration calculating unit 660 can convert the measurement data V (Vx, Vy, Vz) into the magnetic field data B (Bx, By, Bz) as in the following formula.

$$( \begin{matrix}Bx \\ By \\ Bz\end{matrix} ) = {S}^{- 1} \{ ( \begin{matrix}Vx \\ Vy \\ Vz\end{matrix} ) - ( \begin{matrix}Vos , x \\ Vos , y \\ Vos , z\end{matrix} ) \} \qquad [ Formula 5 ]$$

The calibration parameter calculating unit 630 uses environmental magnetic field measurement data to calculate the inverse matrix S⁻¹ of the matrix S, and the offsets (Vos, x, Vos, y, Vos, z), and store them as calibration parameters in the calibration parameter storage unit 640. The calibration calculating unit 660 uses these calibration parameters to convert magnetocardiographic data acquired by the magnetic field acquiring unit 625 into magnetic field measurement data B, and supply the magnetic field measurement data B to the data output unit 680.

In this manner, since each sensor unit 400 has linear characteristics, the calibration calculating unit 660 can use approximately constant coefficients to convert measurement data into magnetic field data. That is, the approximately constant coefficients used by the calibration calculating unit 660 can be determined as a set of calibration parameters using environmental magnetic field data. In addition, the calibration parameter storage unit 640 can store calibration parameters including a coefficient for correcting a cross-axis sensitivity of each of corresponding sensor units 400.

In the example illustrated above, the magnetic field acquiring unit 625 acquires digital measurement data. Instead, the magnetic field acquiring unit 625 may include an AD converter 610, and have a configuration to acquire analog measurement data.

In addition, in the present embodiment, the magnetic field measuring apparatus 10 includes the drive unit 125, and alters the orientation of the magnetic sensor unit 110 automatically under control of the drive control unit 615. Instead, the magnetic field measuring apparatus 10 may not include the drive control unit 615, and drive unit 125, and may have a configuration to alter the orientation of the magnetic sensor unit 110 manually. For example, the magnetic field measuring apparatus 10 may have a configuration in which the magnetic sensor unit 110 can be detached from the head 120, and an administrator of the magnetic field measuring apparatus 10, an laboratory scientist, or the like may turn the detached magnetic sensor unit 110 such that the magnetic sensor unit 110 faces a plurality of directions by manually rotating or turning the magnetic sensor unit 110 or in another manner. The magnetic field acquiring unit 625 may acquire environmental magnetic field measurement data in response to the magnetic sensor unit 110 being turned such that the magnetic sensor unit 110 faces a plurality of directions.

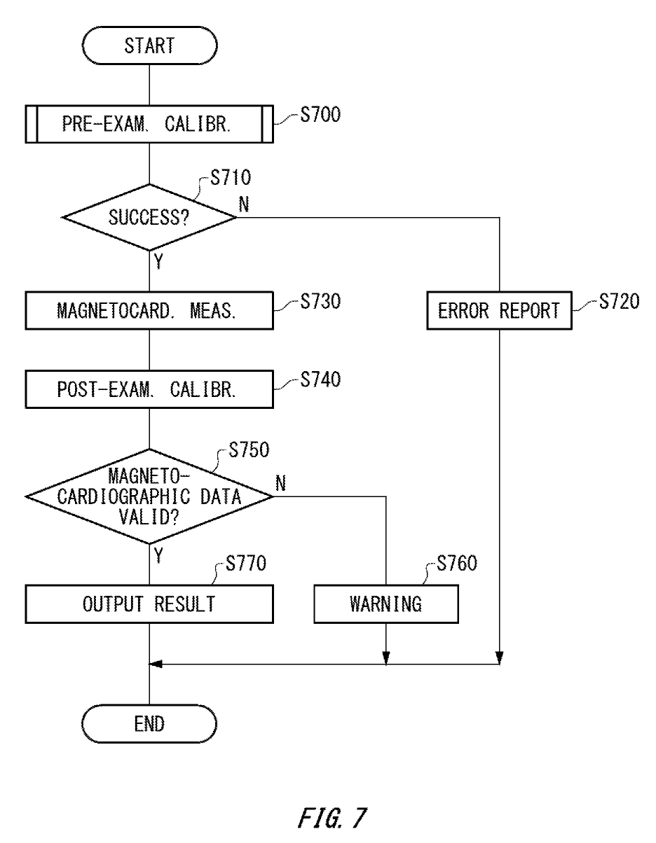

FIG. 7 illustrates an exemplary magnetocardiographic measurement flow for the magnetic field measuring apparatus 10 according to the present embodiment. Instead of or in addition to calibration performed before shipment of the magnetic field measuring apparatus 10 according to the present embodiment, the magnetic field measuring apparatus 10 allows calibration to be performed by acquiring environmental magnetic field measurement data on-site after the magnetic field measuring apparatus 10 is arranged in an examination room or the like where magnetocardiographic measurement is to be performed. Here, that data is acquired on-site means that the data is acquired at a site where a magnetocardiographic measurement apparatus or a calibrating apparatus is located, or magnetocardiographic measurement is to be performed such as an examination room where magnetocardiographic measurement is to be performed. The magnetic field measuring apparatus 10 according to the present embodiment acquires environmental magnetic field measurement data measured before and after magnetocardiographic measurement of a subject, and performs calibration.

The magnetic field measuring apparatus 10 executes the magnetocardiographic measurement flow in this figure in magnetocardiographic measurement of each subject. At S700 (Step 700), the magnetic field measuring apparatus 10 performs calibration before magnetocardiographic measurement (pre-examination calibration), and stores calibration parameters calculated by a calibration parameter calculating unit 630 in a calibration parameter storage unit 640.

At S710, the calibration parameter calculating unit 630 judges whether or not the calibration at S700 succeeded. If the calibration parameter calculating unit 630 judges that the calibration at S700 succeeded, the process proceeds to S730, and if the calibration parameter calculating unit 630 judges that the calibration at S700 failed, the process proceeds to S720. At S720, the calibration parameter calculating unit 630 may use a display device provided to the information processing unit 150 or the like to report an error indicating that the pre-examination calibration failed, and magnetocardiographic measurement cannot be performed at a targeted precision.

At S730, the magnetic field measuring apparatus 10 performs magnetocardiographic measurement of a subject. More specifically, the drive control unit 615 turns the magnetic sensor unit 110 such that the magnetic sensor unit 110 faces a subject. The magnetic sensor array 210 senses a magnetic field at each position where a magnetic sensor cell 220 is provided, and outputs a sensing signal, and a sensor data collecting unit 230 converts a sensing signal from each magnetic sensor cell 220 into digital measurement data. Each magnetic field acquiring unit 625 acquires measurement data obtained through measurement of a magnetic field produced at a measured portion of a subject by the magnetic sensor array 210. The magnetic field acquiring unit 625 may acquire a change of a magnetic field at each position where a magnetic sensor cell 220 is provided as time-series measurement data having data elements (Vx, Vy, Vz) at each timing. The calibration calculating unit 660 uses calibration parameters stored on the calibration parameter storage unit 640, that is, the inverse matrix S⁻¹ and the offsets (Vos, x, Vos, y, Vos, z) for each magnetic sensor cell 220 illustrated with reference to FIG. 6, for example, to calibrate each data element in measurement data as indicated by Formula 5, and calculate measurement data of a magnetic field. A data output unit 680 stores the calculated measurement data of a magnetic field for each magnetic sensor cell 220.

At S740, the magnetic field measuring apparatus 10 performs calibration after magnetocardiographic measurement (post-examination calibration). Here, the drive control unit 615 drives the drive unit 125, and causes the drive unit 125 to alter the orientation of the magnetic sensor array 210. A magnetic field acquiring unit 625 acquires second environmental magnetic field measurement data measured by the magnetic sensor array 210 in response to the magnetic sensor array 210 being turned such that the magnetic sensor array 210 faces a plurality of directions in an environmental magnetic field. Note that in the flow illustrated in this figure, the second environmental magnetic field measurement data at S740 is used for determination about the validity of magnetocardiographic data, and is not for calculating highly precise calibration parameters. Accordingly, the second environmental magnetic field measurement data may have a smaller number of data elements than the first environmental magnetic field measurement data acquired at S700 does.

At S750, a judging units 670 judges whether or not magnetocardiographic data is valid. Since the calibration parameter calculating unit 630 calibrated the sensitivity and offset of each axis for each magnetic sensor cell 220 of the magnetic sensor array 210 at S700, if the sensitivity and offset of each magnetic sensor cell 220 have not changed, each data element (Bx, By, Bz) after calibration of the second environmental magnetic field measurement data is supposed to be distributed on a signal sphere having the magnitude of an environmental magnetic field as its radius. However, if at least one of the sensitivity and offset of a magnetic sensor cell 220 changes, the distribution of data elements (Vx, Vy, Vz) after calibration of the second environmental magnetic field measurement data exhibits a signal sphere distribution not matching the above-mentioned signal sphere or exhibits an ellipsoid distribution. The judging unit 670 judges whether or not magnetocardiographic data is valid according to the degree of difference, from a targeted signal sphere, of the distribution of the calibrated second environmental magnetic field measurement data. If the magnetocardiographic data is judged as valid, the process proceeds to S770, and if the magnetocardiographic data is judged as invalid, the process proceeds to S760. Here, the degree of difference utilized may be, for example, information indicating whether the difference between the sizes of signal spheres obtained from the first and second environmental magnetic field measurement data is within a predetermined range, or information indicating whether the difference between the positions of the centers of the signal spheres is within a predetermined range.

At S760, the judging unit 670 warns a subject, an laboratory scientist, or the like about the fact that the magnetocardiographic data is invalid, by displaying the fact on a display device provided to the information processing unit 150 or the like, for example. The warned subject, laboratory scientist or the like may restart a magnetocardiographic measurement flow by the magnetic field measuring apparatus 10, and the magnetic field measuring apparatus 10 may execute the magnetocardiographic measurement flow in this figure.

At S770, a data output unit 680 outputs magnetocardiographic data. For example, the data output unit 680 may perform display control by which a map indicating the arrangement of the plurality of magnetic sensor cells 220 is displayed on a display device, and the intensity and/or direction of a magnetic field at each magnetic sensor cell 220, or the magnitude and/or direction of the gradient of a magnetic field between each pair of adjacent magnetic sensor cells 220 is/are displayed using colors corresponding to magnetic field intensities or the like and/or arrows or the like corresponding to the directions of the magnetic fields or the like. In addition, the data output unit 680 may perform display control by which the time sequence of magnetocardiographic data is displayed as temporal changes of the above-mentioned map.

The data output unit 680 may calculate the gradients of cardiac magnetic fields using calculation formulae indicated by the following Formulae 6 to 8. Here, Lx, Ly, and Lz represent distances between magnetic sensor cells 220 in the X direction, Y direction, and Z direction, and [i, j, k] indicates the position of a magnetic sensor cell 220.

$$\begin{matrix}( \begin{matrix}Bx \\ By \\ Bz\end{matrix} ) [ i + 1 , j , k ] - ( \begin{matrix}Bx \\ By \\ Bz\end{matrix} ) [ i , j , k ] = ( \begin{matrix}\frac{\partial Bx}{\partial x} \cdot Lx \\ \frac{\partial By}{\partial x} \cdot Lx \\ \frac{\partial Bz}{\partial x} \cdot Lx\end{matrix} ) [ i , j , k ] & [ Formula 6 ] \\ ( \begin{matrix}Bx \\ By \\ Bz\end{matrix} ) [ i , j + 1 , k ] - ( \begin{matrix}Bx \\ By \\ Bz\end{matrix} ) [ i , j , k ] = ( \begin{matrix}\frac{\partial Bx}{\partial y} \cdot Ly \\ \frac{\partial By}{\partial y} \cdot Ly \\ \frac{\partial Bz}{\partial y} \cdot Ly\end{matrix} ) [ i , j , k ] & [ Formula 7 ] \\ ( \begin{matrix}Bx \\ By \\ Bz\end{matrix} ) [ i , j , k + 1 ] - ( \begin{matrix}Bx \\ By \\ Bz\end{matrix} ) [ i , j , k ] = ( \begin{matrix}\frac{\partial Bx}{\partial z} \cdot Lz \\ \frac{\partial By}{\partial z} \cdot Lz \\ \frac{\partial Bz}{\partial z} \cdot Lz\end{matrix} ) [ i , j , k ] & [ Formula 8 ]\end{matrix}$$

Here, each data element (Bx, By, Bz) of magnetocardiographic data corresponding to each magnetic sensor cell 220 includes a component of an environmental magnetic field which is very larger than a component of a cardiac magnetic field. However, the magnetic field measuring apparatus 10 according to the present embodiment makes it possible to precisely calibrate the sensitivity and offset between magnetic sensor cells 220 by calibration as described below. Thereby, in the gradients of cardiac magnetic fields indicated by the above-mentioned Formulae 6 to 8, the components of environmental magnetic fields are sufficiently cancelled between adjacent magnetic sensor cells 220, and highly precise values are obtained.

Note that the data output unit 680 may acquire electrocardiographic signals from an electrocardiograph or the like, and output the electrocardiographic signals in synchronization with magnetocardiographic data. Thereby, the data output unit 680 enables examination using magnetocardiographic data and electrocardiographic signals that are output in synchronization.

The magnetic field measuring apparatus 10 according to the present embodiment performs calibration before and after magnetocardiographic measurement of a subject, and can determine that the validity of magnetocardiographic data is low if a difference is observed between the distributions of environmental magnetic field measurement data before and after the magnetocardiographic measurement. Thereby, the magnetic field measuring apparatus 10 can maintain the precision of magnetocardiographic measurement.

Note that when performing magnetocardiographic measurement of a subject, the magnetic field acquiring unit 625 of the magnetic field measuring apparatus 10 may employ another means for acquiring environmental magnetic field measurement data to be used in calibration of measurement data from the magnetic sensor array 210 measured in the magnetocardiographic measurement. Here, the magnetic field acquiring unit 625 may acquire environmental magnetic field measurement data to be used in calibration for each of a plurality of subjects, or may acquire environmental magnetic field measurement data to be used in calibration in a predetermined cycle (e.g., every hour) or at predetermined timing (at the time of activation of the magnetic field measuring apparatus 10, in the morning, at noon, in the evening, etc.).

In addition, the magnetic field acquiring unit 625 may acquire environmental magnetic field measurement data measured before and/or after magnetocardiographic measurement. In this case, the magnetic field acquiring unit 625 may acquire environmental magnetic field measurement data measured: after magnetocardiographic measurement of a previous subject and before the first magnetocardiographic measurement; and/or after the first magnetocardiographic measurement, and before magnetocardiographic measurement of a succeeding subject, so as to perform calibration for each subject. If environmental magnetic field measurement data is acquired only before or only after magnetocardiographic measurement, the magnetic field measuring apparatus 10 may omit the processes related to judgement of the validity corresponding to S740 to S760 in this figure.

The calibration calculating unit 660 may sequentially perform calibration of measurement data acquired by the magnetic field acquiring unit 625 using calibration parameters immediately after acquisition of each data element of the measurement data, or may sequentially perform calibration of measurement data having been acquired already using calibration parameters in an ex post facto manner. In the latter case, the magnetic field measuring apparatus 10 can also first acquire magnetocardiographic data, then perform the calibration at S700, calculates calibration parameters, and apply the calibration parameters to the magnetocardiographic data.

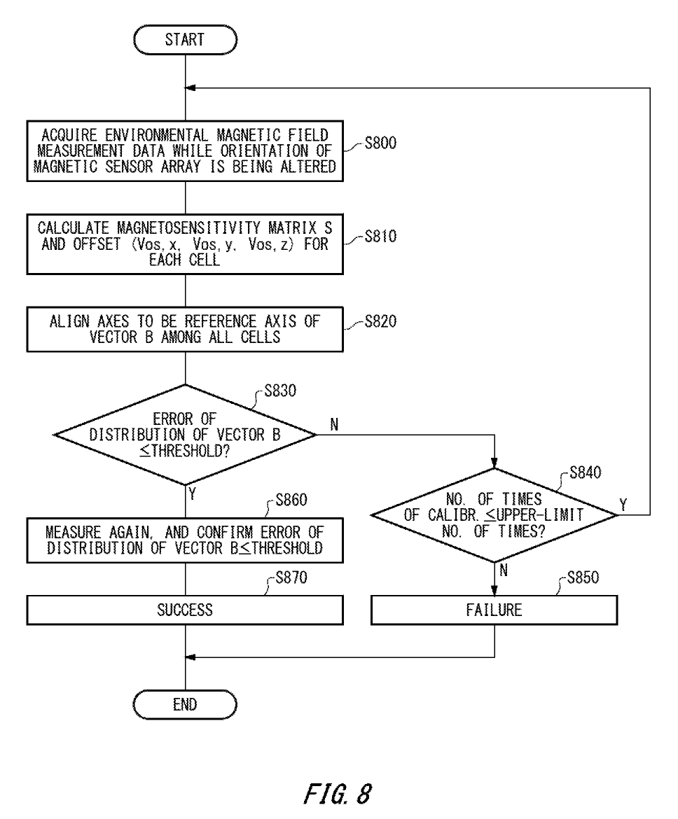

FIG. 8 illustrates an exemplary calibration flow for the magnetic field measuring apparatus 10 according to the present embodiment. At S800, the drive control unit 615 alters the orientation of the magnetic sensor array 210 such that the magnetic sensor array 210 faces a plurality of directions, and each magnetic field acquiring unit 625 acquires environmental magnetic field measurement data measured for each of the plurality of directions. At S810, each calibration parameter calculating unit 630 calculates the magnetosensitivity matrix S and the offsets (Vos, x, Vos, y, Vos, z) for each magnetic sensor cell 220. At S820, the plurality of calibration parameter calculating units 630 align the reference axes of vectors B (Bx, By, Bz) among all the magnetic sensor cells 220.

At S830, for all the magnetic sensor cells 220, the plurality of calibration parameter calculating units 630 judge whether or not errors of the distribution (a signal sphere for triaxial sensors) of the vectors B after calibration of the environmental magnetic field measurement data measured at S800 are no greater than a threshold. Specifically, each calibration parameter calculating unit 630 judges how different the calibrated value of each data element of environmental magnetic field measurement data measured by each magnetic sensor cell 220 is from the spherical surface of a sphere having its center at the origin (0, 0, 0) and the radius corresponding to an environmental magnetic field, that is, judges whether or not an error between the distance of the calibrated value from the origin and the radius corresponding to the environmental magnetic field is no greater than the threshold. Here, the calibration parameter calculating unit 630 may use, as a condition for this judgement, a condition whether all the data elements of environmental magnetic field measurement data satisfy this criteria, or may use, as a condition for this judgement, a condition whether a predetermined ratio (e.g., 99.9%) of data elements satisfies this criteria. If a result of the judgement at S830 is negative, the calibration parameter calculating unit 630 proceeds with the process at S840. If a result of the judgement at S830 is affirmative, the calibration parameter calculating unit 630 proceeds with the process at S860.

At S840, if the number of times of calibration performed at S800 to S820 is no greater than an upper-limit number of times, the calibration parameter calculating unit 630 proceeds with the process at S800, and repeats calibration (S840: Yes). If the number of times of calibration performed exceeds the upper-limit number of times (S840: No), the calibration parameter calculating unit 630 ends the flow in this figure, determining that the calibration failed (S850).

At S860, the magnetic field measuring apparatus 10 measures an environmental magnetic field again, and judges whether or not errors of the distribution of vectors B after calibration of the environmental magnetic field measurement data measured again are no greater than a threshold. This judgement may be the same as the judgement at S830. Upon completion of this confirmation, the calibration parameter calculating unit 630 stores calibration parameters in the calibration parameter storage unit 640, and ends the flow in this figure, determining that the calibration succeeded at S870. Note that if, as a result of the confirmation at S860, errors of the distribution of vectors B after calibration of environmental magnetic field measurement data measured again exceed the threshold, the calibration parameter calculating unit 630 may be configured to proceed with the process at S840.

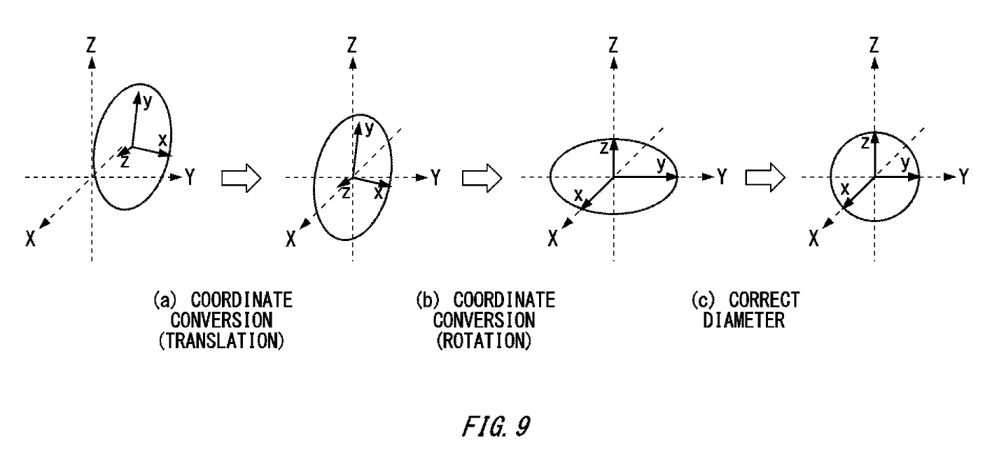

FIG. 9 illustrates exemplary ellipsoid fitting and conversion into a sphere in calibration by the magnetic field measuring apparatus 10 according to the present embodiment. In the ideal state where all the magnetic sensor cells 220 have the same sensitivity in each axial direction, and have no cross-axis sensitivity, and there are no offsets in sensing signals, if each magnetic sensor cell 220 is rotated in a uniform environmental magnetic field, environmental magnetic field measurement data (Vx, Vy, Vz) output by each magnetic sensor cell 220 is distributed on a spherical surface having its center at the origin in a three-dimensional space, and having the radius corresponding to the magnitude of an environmental magnetic field. However, in reality, for causes such as differences in sensitivity in individual axial directions, existence of cross-axis sensitivities, or existence of offsets, the environmental magnetic field measurement data (Vx, Vy, Vz) output by individual magnetic sensor cells 220 are distributed on surfaces of ellipsoids that: have different radii in the individual axial directions due to the differences in sensitivity in the individual axial directions; and are rotated and offset from the origin due to the cross-axis sensitivities.

In view of this, at S810 in FIG. 8, the magnetic field measuring apparatus 10 fits the distribution of the environmental magnetic field measurement data (Vx, Vy, Vz) to an ellipsoid, and calculates correction parameters with which the ellipsoid can be converted into a sphere having its center at the origin. For example, the magnetic field measuring apparatus 10 may perform the process at S810 by a method as illustrated in the following (1) to (3).

(1) Ellipsoid Fitting

Environmental magnetic field measurement data output by each magnetic sensor cell 220 includes N data elements (Vx, Vy, Vz) acquired in the course of altering the azimuth angle and zenith angle of the magnetic sensor array 210. These data elements are distributed on the surface of an ellipsoid for the above-mentioned causes. In view of this, the calibration parameter calculating unit 630 calculates an ellipsoid that fits these data elements.

The general equation of an ellipsoid in a three-dimensional space is as indicated by the following Formula 9. Here, the coefficients a to i are constants.

aX²+bY²+c²+d2XY+e2XZ+f2YZ+g2X+h2Y+i2Z=1 [Formula 9]

For each of N data elements (Vx, Vy, Vz) (N≥9), N row vectors (X², Y², Z², 2XY, 2XZ, 2YZ, 2X, 2Y, 2Z) are calculated, assigning Vx to X, Vy to Y, and Vz to Z.

The above-mentioned N vectors are arrayed in the row direction to generate a matrix D having N rows and nine columns indicated by Formula 10. Here, X, Y, and Z of a k-th data element is referred to as Xₖ, Yₖ, and Zₖ.

$$D = [ \begin{matrix}{X}_{1}^{2} , {Y}_{1}^{2} , {Z}_{1}^{2} , 2 {X}_{1} {Y}_{1} , 2 {X}_{1} {Z}_{1} , 2 {Y}_{1} {Z}_{1} , 2 {X}_{1} , 2 {Y}_{1} , 2 {Z}_{1} \\ {X}_{2}^{2} , {Y}_{2}^{2} , {Z}_{2}^{2} , 2 {X}_{2} {Y}_{2} , 2 {X}_{2} {Z}_{2} , 2 {Y}_{2} {Z}_{2} , 2 {X}_{2} , 2 {Y}_{2} , 2 {Z}_{2} \\ … \\ {X}_{N}^{2} , {Y}_{N}^{2} , {Z}_{N}^{2} , 2 {X}_{N} {Y}_{N} , 2 {X}_{N} {Z}_{N} , 2 {Y}_{N} {Z}_{N} , 2 {X}_{N} , 2 {Y}_{N} , 2 {Z}_{N}\end{matrix} ] \qquad [ Formula 10 ]$$

If a vector v is defined as (a, b, c, d, e, f, g, h, i)^{T}, assigning each of N data elements to Formula 9 gives the following Formula 11. Here, 1[N×1] indicates a matrix having N rows and one column with a component of 1.

Dv=1[N×1] [Formula 11]

Based on Formula 11, the least-squares solution of v can be calculated according to the following Formula 12.

V=(D^{T}D)⁻¹(D^{T}1[N×1]) [Formula 12]

The calibration parameter calculating unit 630 can calculate the constants a to i which are individual elements of v by the ellipsoid fitting illustrated above. By assigning these constants in Formula 9, the calibration parameter calculating unit 630 can obtain the equation of an ellipsoid that fits the distribution of environmental magnetic field measurement data.

(2) Center of Gravity of Ellipsoid

A vector v_{ghi}, a matrix A₄, and a matrix A₃ are defined as in the following Formulae 13 to 15.

$$\begin{matrix}{v}_{ghi} = {[ \begin{matrix}g & h & i\end{matrix} ]}^{T} & [ Formula 13 ] \\ {A}_{4} = [ \begin{matrix}a & d & e & g \\ d & b & f & h \\ e & f & c & i \\ g & h & i & - 1\end{matrix} ] & [ Formula 14 ] \\ {A}_{3} = [ \begin{matrix}a & d & e \\ d & b & f \\ e & f & c\end{matrix} ] & [ Formula 15 ]\end{matrix}$$

At this time, the center of gravity O=[ox, oy, oz]^{T} of an ellipsoid can be calculated according to the following Formula 16.

o=A₃¹v_{ghi} [Formula 16]

A matrix T is defined as in Formula 17.

$$T = [ \begin{matrix}1 & 0 & 0 & 0 \\ 0 & 1 & 0 & 0 \\ 0 & 0 & 1 & 0 \\ ox & oy & o z & 1\end{matrix} ] \qquad [ Formula 17 ]$$

At this time, a matrix B₄ that represents an ellipsoid which corresponds to the ellipsoid represented by the matrix A₄ after its center of gravity is moved to the origin can be calculated according to the following Formula 18.

B₄=TA₄T^{T} [Formula 18]

(3) Rotation of Ellipsoid and Correction of Diameter

Each element of the matrix B₄ calculated according to Formula 18 is represented as in the following Formula 19, and a matrix B₃ based on these element is defined as in Formula 20.

$$\begin{matrix}{B}_{4} = [ \begin{matrix}b 11 & b 12 & b 13 & b 14 \\ b 21 & b 22 & b 23 & b 24 \\ b 31 & b 32 & b 33 & b 34 \\ b 41 & b 42 & b 43 & b 44\end{matrix} ] & [ Formula 19 ] \\ {B}_{3} = \frac{- 1}{b 44} [ \begin{matrix}b 11 & b 12 & b 13 \\ b 21 & b 22 & b 23 \\ b 31 & b 32 & b 33\end{matrix} ] & [ Formula 20 ]\end{matrix}$$

Eigenvalues (λ1, λ2, λ3), and eigenvectors of the matrix B3 are calculated, a matrix having the eigenvalues as diagonal components is defined as Λ, and a matrix formed with the eigenvectors is defined as Q. At this time, the eigenvalues exist in an amount that depends on the size of the diameter of the ellipsoid, and the matrix Q represents the posture of the ellipsoid (i.e., rotation of the axis of the ellipsoid). Accordingly, if the data elements (X, Y, Z) of environmental magnetic field measurement data are assigned in r=[X, Y, Z]^{T}, the calibration parameter calculating unit 630 can convert the data element r on the surface of an ellipsoid into data element d on a spherical surface by: moving the center of gravity of the ellipsoid corresponding to the distribution of environmental magnetic field measurement data to the origin according to the following Formula 21 ((a) in the figure); and rotating the ellipsoid according to the following Formula 22 in such a manner that the axes of the ellipsoid align with the X, Y, and Z axes, and then correcting the diameter ((b), and (c) in the figure).

r′=r−O=[X−ox,Y−oy,Z−oz]^{T} [Formula 21]

d= [symbol] ^{1/2}Q⁻¹r′ [Formula 22]

Note that the conversion according to Formula 22 is conversion into data on a unit spherical surface. Instead, the calibration parameter calculating unit 630 may perform conversion into data on a spherical surface having a radius corresponding to the magnitude of an environmental magnetic field. For example, in such a manner that the sphere after conversion preserves the volume of the ellipsoid, the calibration parameter calculating unit 630 may utilize the eigenvalues (λ1, λ2, λ3) of the matrix B3 to decide the magnitude Ra of the environmental magnetic field according to an equation like the one shown below, and multiply data obtained through conversion according to Formula 22 with the magnitude Ra.

$$Ra = \frac{1}{{( λ1 \times λ2 \times λ3 )}^{\frac{1}{6}}} \qquad [ Formula 23 ]$$

In addition, the magnitude of an environmental magnetic field used by the calibration parameter calculating unit 630 may be measured by installation personnel or the like at the time of installation of the magnetic field measuring apparatus 10 or the like, and set to the magnetic field measuring apparatus 10.

By the method illustrated above as an example, the magnetic field measuring apparatus 10 becomes able to convert environmental magnetic field measurement data from each magnetic sensor cell 220 acquired by each magnetic field acquiring unit 625 into values on a spherical surface. However, the above-mentioned processes may not be enough to eliminate differences among the plurality of magnetic sensor cells 220 in terms of the postures of spheres on which measurement data after calibration of environmental magnetic field measurement data are distributed. In view of this, the magnetic field measuring apparatus 10 aligns the postures of spheres among the plurality of magnetic sensor cells 220 as illustrated in the following (4), for example, at S820 in FIG. 8.

(4) Correction Among Plurality of Magnetic Sensor Cells 220

For a magnetic sensor cell 220 (e.g., a magnetic sensor cell 220 at the position (0, 0, 0)) to serve as a reference cell among the plurality of magnetic sensor cells 220, a matrix in environmental magnetic field measurement data on which the spherical correction corresponding to Formula 22 is performed is defined as Sa, and a measurement data matrix after the spherical correction is performed is defined as Da. For a magnetic sensor cell 220 for which correction is to be performed, a matrix in the environmental magnetic field measurement data on which the spherical correction corresponding to Formula 22 is performed is defined as Sb, and a measurement data matrix after the spherical correction is performed is defined as Db. At this time, Da and Db are each an orthogonally transformed matrix of the other.

The calibration parameter calculating unit 630 calculates a matrix DaDb^{T} which is the product of the matrix Da and the transpose of the matrix Db, and calculates two unitary matrices U and V through singular value decomposition of the matrix DaDb^{T}. At this time, the calibration parameter calculating unit 630 can calculate a matrix R for converting the posture of a signal sphere of the to-be-corrected magnetic sensor cell 220 into the posture of a signal sphere of the reference magnetic sensor cell 220 by R=UV^{T}.

Accordingly, the calibration parameter calculating unit 630 stores Sa of the reference magnetic sensor cell 220 as the inverse matrix S¹ in Formula 5 in the calibration parameter storage unit 640, and stores RSb of the to-be-corrected magnetic sensor cell 220 as the inverse matrix S⁻¹ in the calibration parameter storage unit 640. According to the above-mentioned (1) to (4), for the plurality of magnetic sensor cells 220, the calibration parameter calculating unit 630 can calculate correction parameters for adjusting the sensitivities of individual axes, eliminating the influence of cross-axis sensitivities, correcting offsets, and aligning the postures among individual magnetic sensor cells 220. Note that by measuring in advance already known magnetic fields to be an orthogonal coordinate system, a posture relative to the orthogonal coordinate system may be decided in advance, and the posture of each magnetic sensor cell 220 may be corrected.

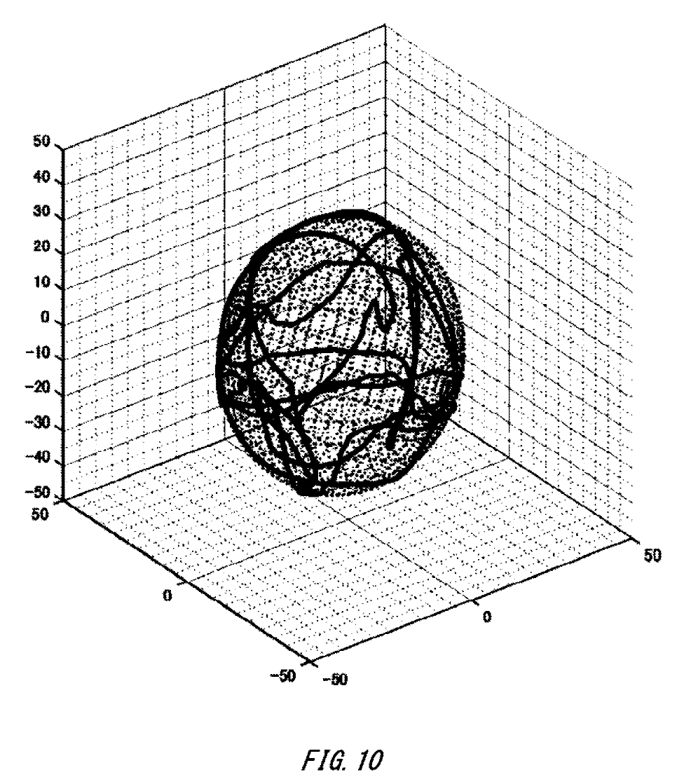

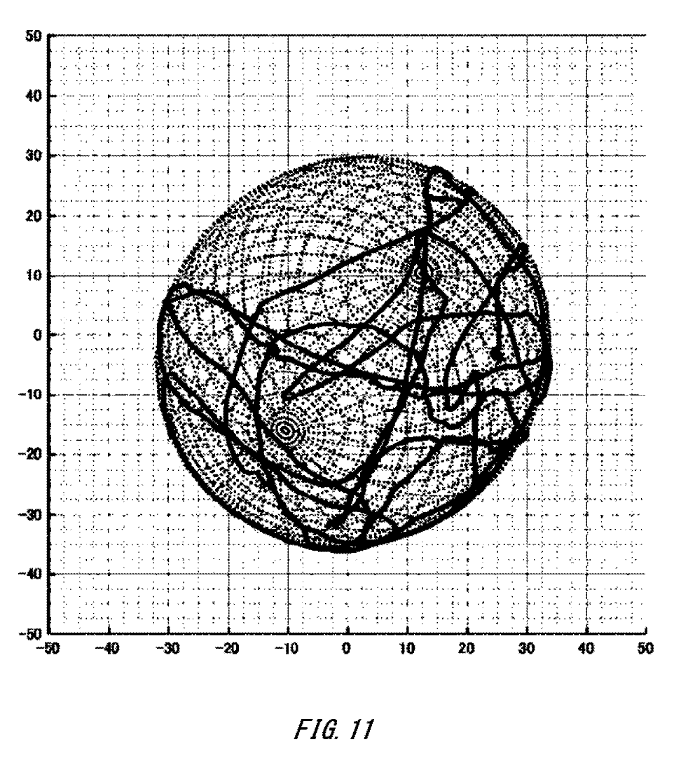

FIG. 10 illustrates exemplary measurement data before conversion into a sphere by the magnetic field measuring apparatus 10 according to the present embodiment. FIG. 11 illustrates a projection, on the YZ plane, of the measurement data before conversion into a sphere illustrated in FIG. 10. In these figures, measurement data measured using triaxial magnetic sensors in a static magnetic field environment of about 30 μT is indicated by solid lines, and an ellipsoid that fits the measurement data is indicated by dotted lines for reference.

In these figures, the unit of values in the x-, y-, and z-axis directions is μT. At S800 and S860 in FIG. 8, and at S740 in FIG. 7, the drive unit 125 may turn the magnetic sensor array 210 such that the magnetic sensor array 210 faces a plurality of directions under control of the drive control unit 615. Every time the drive unit 125 turns the magnetic sensor array 210 such that the magnetic sensor array 210 faces a certain direction, the drive unit 125 may make the magnetic sensor array 210 stationary temporarily, and, in this manner, the magnetic field acquiring unit 625 may acquire environmental magnetic field measurement data in response to the magnetic sensor array 210 being turned by the drive unit 125 such that the magnetic sensor array 210 faces the plurality of directions.

Instead, the drive unit 125 may continuously alter the orientation of the magnetic sensor array 210. For example, the drive unit 125 may continuously alter the zenith angle and azimuth angle of the magnetic sensor array 210 randomly or in another manner as illustrated in these figures. While the orientation of the magnetic sensor array 210 is being altered, the magnetic field acquiring unit 625 may sample data elements of environmental magnetic field measurement data at predetermined timing, that is, at any timing, for example, in a constant time cycle, or every time the zenith angle or azimuth angle is altered by a certain degree.

Here, even if the drive unit 125 turns the magnetic sensor array 210 such that the magnetic sensor array 210 faces completely different directions at every instance of calibration, the calibration parameter calculating unit 630 can perform ellipsoid fitting appropriately as long as the number of data elements of measurement data is sufficiently large.

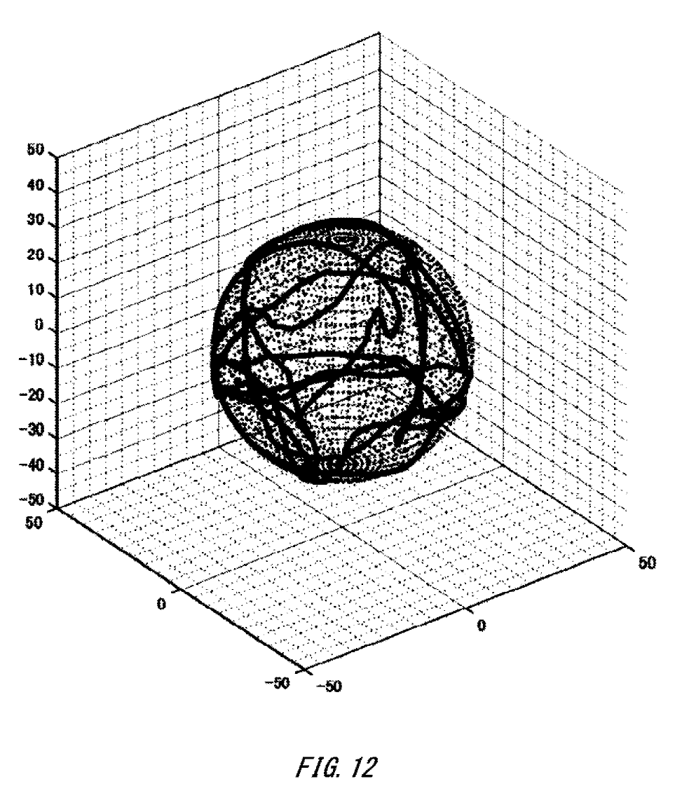

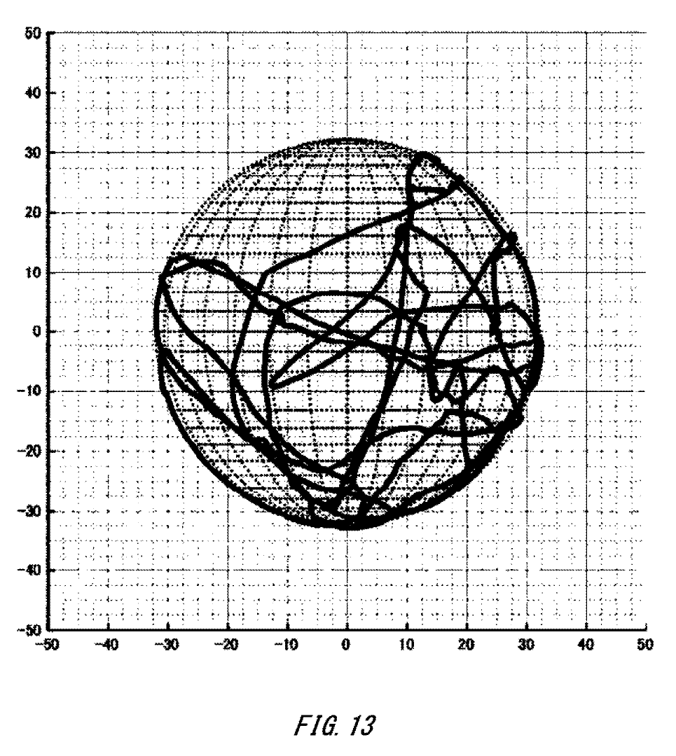

FIG. 12 illustrates exemplary measurement data after conversion into a sphere by the magnetic field measuring apparatus 10 according to the present embodiment. FIG. 13 illustrates a projection, on the YZ plane, of the measurement data after conversion into a sphere illustrated in FIG. 12. These figures illustrate data after a series of unconverted measurement data illustrated in FIG. 10 and FIG. 11 is converted into data on a spherical surface using the method illustrated with reference to FIG. 9.

As illustrated in these figures, it can be known that, by performing ellipsoid fitting and conversion into a sphere by the method illustrated with reference to FIG. 9, the calibration parameter calculating unit 630 could convert environmental magnetic field measurement data acquired by the magnetic field acquiring unit 625 into measurement data on the spherical surface. In particular, there are directions in a relatively wide range on the upper to left sides in FIG. 13 which the magnetic sensor array 210 has not faced. It can be known that even in such a case, the calibration parameter calculating unit 630 can convert the acquired environmental magnetic field measurement data into measurement data on the spherical surface by the method illustrated with reference to FIG. 9. Accordingly, the drive unit 125 may have a blind spot which the magnetic sensor array 210 does not face. The range of the blind spot may be, for example, a range smaller than a semisphere as seen from the center of the sphere, or may be a range extending less than 90 degrees as seen from the center of the sphere, and these conditions may be met for only one of two orthogonal directions on the spherical surface. In addition, the area ratio of the range of the blind spot portion to the entire surface of the sphere may be less than ½, less than ¼, less than ⅛, or the like.

Note that the magnetic sensor unit 110 may further include a triaxial acceleration sensor. If a triaxial acceleration sensor is included, an acceleration vector representing the direction of gravity is obtained corresponding to the posture of the magnetic sensor unit 110. The acceleration vector, and a vector of an environmental magnetic field indicating a certain direction obtained by the magnetic sensor unit 110 can be used to define an orthogonal coordinate system by a vector product, with the ground being used as its plane, for example. Therefore, the posture of the magnetic sensor unit 110 relative to the ground can be decided by calculation. Because of this, even if the magnetic sensor unit 110 is assuming any posture, posture correction of magnetocardiographic data relative to the orthogonal coordinate system can be performed based on a matrix representing the posture obtained through calculation. This means that even if the magnetic sensor unit 110 is moved for some cause, or a subject of magnetocardiographic measurement puts the magnetic sensor unit 110 on his/her body, and changes his/her posture, magnetocardiographic data can be measured correctly relative to the orthogonal coordinate system. Note that the posture relative to the ground has to be obtained only for a magnetic sensor cell 220 (e.g., the magnetic sensor cell 220 at the position (0, 0, 0)) to serve as a reference cell among the plurality of magnetic sensor cells 220, and only one acceleration sensor is required. Note that the acceleration sensor may be included in the magnetic sensor array 210 constituting the magnetic sensor unit 110.

Note that in the present embodiment explained, each magnetic sensor cell 220 is a triaxial magnetic sensor. Since if each magnetic sensor cell 220 is a triaxial magnetic sensor, components of a vector of a magnetic field in a three dimensional space are measured directly, measurement is possible in any posture, correction of cross-axis sensitivities is also possible, and thus it is suitable for improvement of calibration precision, and measurement precision after calibration. Instead, the magnetic sensor cells 220 may be biaxial magnetic sensors, uniaxial magnetic sensors, or the like.

If each magnetic sensor cell 220 is a biaxial magnetic sensor, the drive unit 125 may alter the orientation of the magnetic sensor array 210 such that the magnetic sensor array 210 faces a plurality of directions by rotating the magnetic sensor array 210 about an axis orthogonal to two magnetosensitive axis directions or in another manner. For example, if each magnetic sensor cell 220 has a magnetic sensor that senses magnetic fields in the X-axis direction and Z-axis direction, the drive unit 125 may rotate the magnetic sensor array 210 about the Y axis.

Thereby, as the distribution of environmental magnetic field measurement data, the calibration parameter calculating unit 630 can obtain a distribution on an oval surface on the XZ plane, instead of an ellipsoid in the case of a triaxial magnetic sensor. The calibration parameter calculating unit 630 can calculate calibration parameters for converting the oval into a circle having its center at the origin, and aligning the postures of the circles among the magnetic sensor cells 220.

Note that each magnetic sensor cell 220 may have two magnetosensitive axes in two axial (e.g., the X-axis and Y-axis in FIG. 1) directions that are parallel to a surface of the magnetic sensor unit 110 at which it faces a subject. Instead, each magnetic sensor cell 220 may have a magnetosensitive axis in one axial (e.g., the X-axis or Y-axis in FIG. 1) direction that is parallel to a surface of the magnetic sensor unit 110 at which it faces a subject, and a magnetosensitive axis in one axial (e.g., the Z-axis in FIG. 1) direction that is orthogonal to the surface of the magnetic sensor unit 110 at which it faces the subject.

In addition, if each magnetic sensor cell 220 is a uniaxial magnetic sensor, the drive unit 125 may alter the orientation of the magnetic sensor array 210 such that the magnetic sensor array 210 faces a plurality of directions by rotating the magnetic sensor array 210 about an axis orthogonal to a magnetosensitive axis direction or in another manner. For example, if each magnetic sensor cell 220 has a magnetic sensor that senses a magnetic field in the Z-axis direction, the drive unit 125 may rotate the magnetic sensor array 210 about the Y axis.

Thereby, as the distribution of environmental magnetic field measurement data, the calibration parameter calculating unit 630 can obtain a graph representing a relationship between the rotation angle of the drive unit 125 and environmental magnetic field measurement data measured by the magnetic sensor cell 220, instead of an ellipsoid as in the case of triaxial magnetic sensors. Then, the calibration parameter calculating unit 630 can calculate calibration parameters for converting this environmental magnetic field measurement data into a sine wave or a cosine wave having its center at the value of 0, and making the amplitudes and phases of the sine waves or the like matched among individual magnetic sensor cells 220. Here, if the rotation angle of a magnetic sensor cell 220 rotated by the drive unit 125 can be sensed additionally, the calibration parameter calculating unit 630 may use, as an offset included in correction parameters, the average value of environmental magnetic field measurement data in the case where the magnetic sensor cell 220 faces a certain direction, and environmental magnetic field measurement data in the case where the magnetic sensor cell 220 faces the opposite direction different from the above-mentioned certain direction by 180 degrees.

Note that each magnetic sensor cell 220 may have a magnetosensitive axis in one axial (e.g., the X-axis or Y-axis in FIG. 1) direction that is parallel to a surface of the magnetic sensor unit 110 at which it faces a subject, and may have a magnetosensitive axis in one axial (e.g., the Z-axis in FIG. 1) direction that is orthogonal to the surface of the magnetic sensor unit 110 at which it faces the subject.

In addition, the magnetic sensor array 210 may be a hybrid array. That is, some of the magnetic sensor cells 220 of the magnetic sensor array 210 may be triaxial magnetic sensor cells, and the rest may biaxial magnetic sensor cells or uniaxial magnetic sensor cells. The hybrid magnetic sensor array 210 utilizes the triaxial magnetic sensor cells as sensors for performing reference about an environmental magnetic field (reference sensors). In addition to oscillating the magnetic sensor array 210 for calibration of the triaxial magnetic sensor cells, the magnetic field measuring apparatus 10 oscillates some uniaxial, and biaxial magnetic sensor cells. The oscillation of the uniaxial, and biaxial sensor cells provides measurement data for calibration of the triaxial sensor cells. In the case of such a hybrid magnetic sensor array, environmental magnetic field measurement data is measured as three dimensional vector components by the triaxial magnetic sensor cells, but the uniaxial, and biaxial sensor cells can measure only one-dimensional, and two-dimensional vector components. In view of this, vector components of the remaining dimensions are estimated from three dimensional vector components measured by the triaxial magnetic sensor cells.

As as illustrated in FIG. 1 for example, the XZ plane is defined as the ground plane, the uniaxial magnetic sensor cells are arranged along the Z axis, and magnetocardiographic measurement is performed along the Z axis. It is supposed that at this time, the environmental magnetic field Bfull is tilted by 45 degrees from the Z axis along the YZ plane. This environmental magnetic field intensity and slope can be determined by utilizing the triaxial magnetic sensor cells. Then, in this case, the magnitude of an environmental magnetic field sensed by the uniaxial sensor cells having the main axis sensitivity in the Z-axis direction can be estimated to be) Bfull×cos(45°). By subtracting this from measurements of the uniaxial sensor cells, a measurement result from which the influence of the environmental magnetic field is eliminated can be obtained.

In addition, similarly, it is supposed that the biaxial magnetic sensor cells capable of sensing a magnetic field on the XY plane are arranged along the XY plane, and magnetocardiographic measurement is performed on the XY plane. It is supposed that at this time, the environmental magnetic field Bfull is tilted by 45 degrees from the Z axis along the YZ plane. This environmental magnetic field intensity and slope can be determined by utilizing the triaxial magnetic sensor cells. Then, in this case, the magnitude of an environmental magnetic field sensed by magnetic sensor cells having the main axis sensitivity in the Y-axis direction among magnetic sensors in the biaxial magnetic sensor cell can be estimated to be Bfull×cos(45°). In addition, the magnitude of an environmental magnetic field sensed by magnetic sensors having the main axis sensitivity in the X-axis direction can be estimated to be 0. By subtracting this from measurements of the biaxial sensor cells, a measurement result from which the influence of the environmental magnetic field is eliminated can be obtained.

Note that in the above-mentioned explanation, an exemplary configuration of the sensor data processing unit 600 in the present embodiment is illustrated in FIG. 6. However, the configuration of the sensor data processing unit 600 is not limited to this. For example, the sensor data processing unit 600 may include a storage unit (hereinafter, referred to as a storage unit for temporary storage) for temporarily storing data acquired by the judging unit 670. The storage unit for temporary storage may include a first environmental magnetic field measurement data storage unit, and a second environmental magnetic field measurement data storage unit, for example. The first environmental magnetic field measurement data storage unit temporarily stores first environmental magnetic field measurement data acquired by a magnetic field acquiring unit 625. In addition, the second environmental magnetic field measurement data storage unit temporarily stores second environmental magnetic field measurement data acquired by the magnetic field acquiring unit 625. In addition, these storage units for temporary storage may be storages independent of a calibration parameter storage unit 640, or may be part of the calibration parameter storage unit 640. In addition, a judging unit 670 may acquire data directly from the storage units for temporary storage (the first environmental magnetic field measurement data storage unit, and second environmental magnetic field measurement data storage unit), or may acquire data as calibrated data via a calibration calculating unit 660. For example, the first environmental magnetic field measurement data may be stored on the first environmental magnetic field measurement data storage unit, and the second environmental magnetic field measurement data may be stored on the second environmental magnetic field measurement data storage unit. Then, the judging unit 670 may perform judgement by retrieving the first environmental magnetic field measurement data from the first environmental magnetic field measurement data storage unit, and acquiring first calibration data calibrated at the calibration calculating unit 660, and retrieving the second environmental magnetic field measurement data from the second environmental magnetic field measurement data storage unit, and acquiring second calibration data calibrated at the calibration calculating unit 660. Prior to this, calibration parameters may be calculated in advance from the first environmental magnetic field measurement data at a calibration parameter calculating unit 630, and, at the calibration calculating unit 660, the calibration parameters may be utilized to calculate second calibration data from the second environmental magnetic field measurement data.

Various embodiments of the present invention may be described with reference to flowcharts and block diagrams whose blocks may represent (1) steps of processes in which operations are performed or (2) sections of apparatuses responsible for performing operations. Certain steps and units may be implemented by dedicated circuitry, programmable circuitry supplied with computer-readable instructions stored on computer-readable media, and/or processors supplied with computer-readable instructions stored on computer-readable media. Dedicated circuitry may include digital and/or analog hardware circuits and may include integrated circuits (IC) and/or discrete circuits. Programmable circuitry may include reconfigurable hardware circuits comprising logical AND, OR, XOR, NAND, NOR, and other logical operations, flip-flops, registers, memory elements, etc., such as field-programmable gate arrays (FPGA), programmable logic arrays (PLA), etc.

Computer-readable media may include any tangible device that can store instructions for execution by a suitable device, such that the computer-readable medium having instructions stored therein comprises an article of manufacture including instructions which can be executed to create means for performing operations specified in the flowcharts or block diagrams. Examples of computer-readable media may include an electronic storage medium, a magnetic storage medium, an optical storage medium, an electromagnetic storage medium, a semiconductor storage medium, etc. More specific examples of computer-readable media may include a floppy (registered trademark) disk, a diskette, a hard disk, a random access memory (RAM), a read-only memory (ROM), an erasable programmable read-only memory (EPROM or Flash memory), an electrically erasable programmable read-only memory (EEPROM), a static random access memory (SRAM), a compact disc read-only memory (CD-ROM), a digital versatile disk (DVD), a BLU-RAY (registered trademark) disc, a memory stick, an integrated circuit card, etc.

Computer-readable instructions may include assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, JAVA (registered trademark), C++, etc., and conventional procedural programming languages, such as the “C” programming language or similar programming languages.

Computer-readable instructions may be provided to a processor of a general purpose computer, special purpose computer, or other programmable data processing apparatus, or to programmable circuitry, locally or via a local area network (LAN), wide area network (WAN) such as the Internet, etc., to execute the computer-readable instructions to create means for performing operations specified in the flowcharts or block diagrams. Examples of processors include computer processors, processing units, microprocessors, digital signal processors, controllers, microcontrollers, etc.

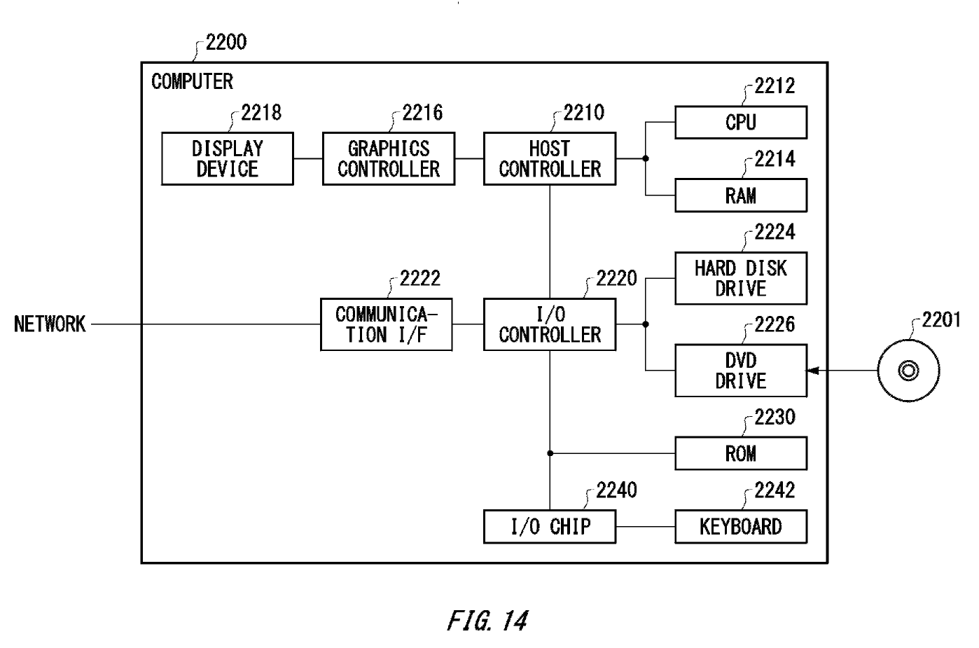

FIG. 14 shows an example of a computer 2200 in which aspects of the present invention may be wholly or partly embodied. A program that is installed in the computer 2200 can cause the computer 2200 to function as or perform operations associated with apparatuses of the embodiments of the present invention or one or more sections thereof, and/or cause the computer 2200 to perform processes of the embodiments of the present invention or steps thereof. Such a program may be executed by the CPU 2212 to cause the computer 2200 to perform certain operations associated with some or all of the blocks of flowcharts and block diagrams described herein.

The computer 2200 according to the present embodiment includes a CPU 2212, a RAM 2214, a graphics controller 2216, and a display device 2218, which are mutually connected by a host controller 2210. The computer 2200 also includes input/output units such as a communication interface 2222, a hard disk drive 2224, a DVD-ROM drive 2226 and an IC card drive, which are connected to the host controller 2210 via an input/output controller 2220. The computer also includes legacy input/output units such as a ROM 2230 and a keyboard 2242, which are connected to the input/output controller 2220 through an input/output chip 2240.

The CPU 2212 operates according to programs stored in the ROM 2230 and the RAM 2214, thereby controlling each unit. The graphics controller 2216 obtains image data generated by the CPU 2212 on a frame buffer or the like provided in the RAM 2214 or in itself, and causes the image data to be displayed on the display device 2218.

The communication interface 2222 communicates with other electronic devices via a network. The hard disk drive 2224 stores programs and data used by the CPU 2212 within the computer 2200. The DVD-ROM drive 2226 reads the programs or the data from the DVD-ROM 2201, and provides the hard disk drive 2224 with the programs or the data via the RAM 2214. The IC card drive reads programs and data from an IC card, and/or writes programs and data into the IC card.

The ROM 2230 stores therein a boot program or the like executed by the computer 2200 at the time of activation, and/or a program depending on the hardware of the computer 2200. The input/output chip 2240 may also connect various input/output units via a parallel port, a serial port, a keyboard port, a mouse port, and the like to the input/output controller 2220.

A program is provided by computer-readable media such as the DVD-ROM 2201 or the IC card. The program is read from the computer-readable media, installed into the hard disk drive 2224, RAM 2214, or ROM 2230, which are also examples of computer-readable media, and executed by the CPU 2212. The information processing described in these programs is read into the computer 2200, resulting in cooperation between a program and the above-mentioned various types of hardware resources. An apparatus or method may be constituted by realizing the operation or processing of information in accordance with the usage of the computer 2200.

For example, when communication is performed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

In addition, the CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, etc., and perform various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may perform various types of processing on the data read from the RAM 2214, which includes various types of operations, processing of information, condition judging, conditional branch, unconditional branch, search/replace of information, etc., as described throughout this disclosure and designated by an instruction sequence of programs, and writes the result back to the RAM 2214. In addition, the CPU 2212 may search for information in a file, a database, etc., in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, and read the attribute value of the second attribute stored in the entry, thereby obtaining the attribute value of the second attribute associated with the first attribute satisfying the predetermined condition.

The above-explained program or software modules may be stored in the computer-readable media on or near the computer 2200. In addition, a recording medium such as a hard disk or a RAM provided in a server system connected to a dedicated communication network or the Internet can be used as the computer-readable media, thereby providing the program to the computer 2200 via the network.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

What is claimed is:

1. A magnetocardiographic measurement apparatus comprising:
a magnetic sensor array;
a magnetic field acquiring unit that acquires environmental magnetic field measurement data measured by the magnetic sensor array in response to the magnetic sensor array being turned such that the magnetic sensor array faces a plurality of directions in an environmental magnetic field;
a calibration parameter calculating unit that uses the environmental magnetic field measurement data to calculate a calibration parameter for calibrating measurement data measured by the magnetic sensor array in magnetocardiographic measurement of a subject;
a calibration parameter storage unit that stores the calculated calibration parameter;
a calibration calculating unit that uses the stored calibration parameter to calibrate the measurement data; and
a data output unit that outputs the calibrated measurement data, wherein
the magnetic sensor array has a plurality of magnetic sensor cells that are arrayed three-dimensionally, and are each capable of sensing a magnetic field in three axial directions.

2. The magnetocardiographic measurement apparatus according to claim 1, wherein the magnetic field acquiring unit acquires the environmental magnetic field measurement data on-site.

3. The magnetocardiographic measurement apparatus according to claim 1, wherein if first magnetocardiographic measurement is performed on a subject, the magnetic field acquiring unit acquires the environmental magnetic field measurement data to be used in calibration of measurement data measured in the first magnetocardiographic measurement by the magnetic sensor array.

4. The magnetocardiographic measurement apparatus according to claim 3, wherein the magnetic field acquiring unit acquires the environmental magnetic field measurement data measured before and/or after the first magnetocardiographic measurement.

5. The magnetocardiographic measurement apparatus according to claim 4, wherein the magnetic field acquiring unit acquires the environmental magnetic field measurement data measured:
after magnetocardiographic measurement of a previous subject and before the first magnetocardiographic measurement; and/or
after the first magnetocardiographic measurement, and before magnetocardiographic measurement of a succeeding subject.

6. The magnetocardiographic measurement apparatus according to claim 4, wherein
the magnetic field acquiring unit acquires first environmental magnetic field measurement data measured before the first magnetocardiographic measurement, and second environmental magnetic field measurement data measured after the first magnetocardiographic measurement,
the calibration parameter calculating unit uses the first environmental magnetic field measurement data to calculate the calibration parameter, and
the magnetocardiographic measurement apparatus further comprises a judging unit that uses the second environmental magnetic field measurement data to judge validity of measurement data measured in the first magnetocardiographic measurement by the magnetic sensor array.

7. The magnetocardiographic measurement apparatus according to claim 6, wherein the second environmental magnetic field measurement data has a smaller number of data elements than the first environmental magnetic field measurement data does.

8. The magnetocardiographic measurement apparatus according to claim 1, further comprising a drive unit that alters an orientation of the magnetic sensor array, wherein
the magnetic field acquiring unit acquires the environmental magnetic field measurement data in response to the magnetic sensor array being turned by the drive unit such that the magnetic sensor array faces the plurality of directions.

9. The magnetocardiographic measurement apparatus according to claim 8, wherein
the drive unit alters the orientation of the magnetic sensor array continuously, and
the magnetic field acquiring unit samples a data element in the environmental magnetic field measurement data at predetermined timing while the orientation of the magnetic sensor array is being altered.

10. The magnetocardiographic measurement apparatus according to claim 8, wherein the drive unit alters a zenith angle and an azimuth angle of the magnetic sensor array.

11. The magnetocardiographic measurement apparatus according to claim 8, wherein the drive unit alters the orientation of the magnetic sensor array relative to a head of the magnetocardiographic measurement apparatus, the head being configured to make the magnetic sensor array face a subject.

12. The magnetocardiographic measurement apparatus according to claim 8, wherein the drive unit alters the orientation of the magnetic sensor array after the magnetic sensor array is detached from a head of the magnetocardiographic measurement apparatus, the head being configured to make the magnetic sensor array face a subject.

13. A calibration method by which a magnetocardiographic measurement apparatus calibrates measurement data in magnetocardiographic measurement, the calibration method comprising:
acquiring, by the magnetocardiographic measurement apparatus, environmental magnetic field measurement data measured by a magnetic sensor array in response to the magnetic sensor array being turned such that the magnetic sensor array faces a plurality of directions in an environmental magnetic field, the magnetic sensor array having a plurality of magnetic sensor cells that are arrayed three-dimensionally, and are each capable of sensing a magnetic field in three axial directions;
calculating, by the magnetocardiographic measurement apparatus using the environmental magnetic field measurement data, a calibration parameter for calibrating measurement data measured by the magnetic sensor array in magnetocardiographic measurement of a subject;
storing, in the magnetocardiographic measurement apparatus, the calculated calibration parameter; and
calibrating, by the magnetocardiographic measurement apparatus, the measurement data using the stored calibration parameter.

14. The calibration method according to claim 13, wherein, in the acquisition of the environmental magnetic field measurement data, if first magnetocardiographic measurement is performed on a subject, the magnetocardiographic measurement apparatus acquires the environmental magnetic field measurement data to be used in calibration of measurement data measured in the first magnetocardiographic measurement by the magnetic sensor array.

15. The calibration method according to claim 14, wherein in the acquisition of the environmental magnetic field measurement data, the magnetocardiographic measurement apparatus acquires the environmental magnetic field measurement data measured before and/or after the first magnetocardiographic measurement.

16. The calibration method according to claim 13, further comprising altering an orientation of the magnetic sensor array by the magnetocardiographic measurement apparatus, wherein
in the acquisition of the environmental magnetic field measurement data, the magnetocardiographic measurement apparatus acquires the environmental magnetic field measurement data in response to the magnetic sensor array being turned such that the magnetic sensor array faces the plurality of directions.

17. The calibration method according to claim 16, wherein
in the alteration of the orientation of the magnetic sensor array, the magnetocardiographic measurement apparatus alters the orientation of the magnetic sensor array continuously, and
in the acquisition of the environmental magnetic field measurement data, the magnetocardiographic measurement apparatus samples a data element in the environmental magnetic field measurement data at predetermined timing while the orientation of the magnetic sensor array is being altered.

18. The calibration method according to claim 16, wherein in the alteration of the orientation of the magnetic sensor array, the magnetocardiographic measurement apparatus alters a zenith angle and an azimuth angle of the magnetic sensor array.

19. A recording medium having recorded thereon a calibration program that, upon being executed by a computer, causes the computer to function as:
a magnetic field acquiring unit that acquires environmental magnetic field measurement data measured by a magnetic sensor array in response to the magnetic sensor array being turned such that the magnetic sensor array faces a plurality of directions in an environmental magnetic field, the magnetic sensor array having a plurality of magnetic sensor cells that are arrayed three-dimensionally, and are each capable of sensing a magnetic field in three axial directions;
a calibration parameter calculating unit that uses the environmental magnetic field measurement data to calculate a calibration parameter for calibrating measurement data measured by the magnetic sensor array in magnetocardiographic measurement of a subject;
a calibration parameter storage unit that stores the calculated calibration parameter; and
a calibration calculating unit that uses the stored calibration parameter to calibrate the measurement data.
